(19) United States
(12) Patent Application Publication
(10) Pub. No.: US 2022/0122721 A1
(43) Pub. Date: Apr. 21, 2022

(54) MACHINE LEARNING METHODS FOR ANALYZING USER INFORMATION TO MATCH USERS WITH APPROPRIATE THERAPISTS AND TREATMENT FACILITIES

(71) Applicant: Spa Space App Inc., Atlanta, GA (US)

(72) Inventor: Ilana ALBERICO, Atlanta, GA (US)

(21) Appl. No.: 17/507,399

(22) Filed: Oct. 21, 2021

Related U.S. Application Data
(60) Provisional application No. 63/094,530, filed on Oct. 21, 2020.

(51) Int. Cl. G16H 40/20; G06F 16/2457; G16H 50/70; G16H 50/20

(52) U.S. Cl. CPC G16H 40/20; G16H 50/20; G16H 50/70; G06F 16/24578

(57) ABSTRACT

A system can include a computing device operatively connected to a data store. The computing device can receive a request to generate a scheduling object from a network address, the request comprising a subset of criteria. The computing device can determine entries based on the subset of criteria. The computing device can generate a respective match score for each of the entries and generate a ranking of the entries based on each corresponding match score. The computing device can determine one or more top-ranked entries based on the ranking and serve the one or more top-ranked entries to the particular network address. The computing device can receive a selection of a particular entry and, in response, generate the scheduling object based on the particular entry. The computing device can transmit the scheduling object to the particular network address and store the scheduling object at the data store.

### CROSS REFERENCE TO RELATED APPLICATIONS

This application claims the benefit of and priority to U.S. Application No. 63/094,530, filed Oct. 21, 2020, entitled “APPARATUS AND METHODS FOR ANALYZING USER INFORMATION TO MATCH USERS WITH APPROPRIATE THERAPISTS AND TREATMENT FACILITIES,” the disclosure of which is incorporated herein by reference in its entirety.

### TECHNICAL FIELD

The present application generally relates to the field of information technologies, and in particular to methods and apparatus for analyzing user information to match users with appropriate therapists and treatment facilities.

### BACKGROUND

In some embodiments, matching and scheduling apparatus and methods are useful in many applications including the therapeutics industry. Some known apparatus or methods for scheduling an event upon a user request, however, do not include a secure and reliable platform to consider user information, therapist information, and facility information for scheduling an event (e.g., a treatment session).

Therefore, a need exists for apparatus and methods for efficiently matching information about the user with the therapist information and the facility information.

### BRIEF SUMMARY OF THE DISCLOSURE

In some embodiments, an apparatus receives a request from a user to schedule an event. The apparatus further receives data including information about the user, a set of indications of expertise of a set of therapists, a set of indications of amenities in a set of facilities, a first time schedule associated with the user, a second time schedule associated with the set of therapists, and a third time schedule associated with the set of facilities. The apparatus further detects a set of entries, each entry indicating a therapist from the set of therapists, a facility from the set of facilities, and a calculated matching score in response to the request. The apparatus further detects a set of available times common to the user, the therapist, and the facility in response to the request. The apparatus further ranks at least one of the set of therapists and the set of facilities based on feedback from the user after the event.

According to a first aspect, a method, comprising: A) receiving, via at least one computing device, a request to generate a scheduling object from a particular network address, the request comprising a subset of a plurality of criteria; B) determining, via the at least one computing device, a plurality of entries based on the subset of the plurality of criteria; C) generating, via the at least one computing device, at least one respective match score for each of the plurality of entries; D) generating, via the at least one computing device, a ranking of the plurality of entries based on each corresponding one of the at least one respective match score; E) determining, via the at least one computing device, one or more top-ranked entries based on the ranking; F) serving, via the at least one computing device, the one or more top-ranked entries to the particular network address; G) receiving, via the at least one computing device, a selection of a particular entry of the one or more top-ranked entries; H) in response to the selection, generating, via the at least one computing device, the scheduling object based on the particular entry; I) transmitting, via the at least one computing device, the scheduling object to the particular network address; and J) storing, via the at least one computing device, the scheduling object in a storage device associated with the at least one computing device.

According to a further aspect, the method of the first aspect or any other aspect, wherein the plurality of criteria comprises one or more of a time interval, a location, one or more treatment preferences, and one or more product preferences.

According to a further aspect, the method of the first aspect or any other aspect, wherein the one or more treatment preferences comprise at least one treatment preference selected from the group consisting of: practitioner age, practitioner gender, treatment, treatment intensity, and treatment duration.

According to a further aspect, the method of the first aspect or any other aspect, wherein the subset of the plurality of criteria comprises a location of the at least one computing device.

According to a further aspect, the method of the first aspect or any other aspect, further comprising, in response to detecting an access event, determining a location of the at least one computing device, wherein at least one of the plurality of criteria is based on the location.

According to a further aspect, the method of the first aspect or any other aspect, wherein determining the plurality of entries comprises identifying, for each of the plurality of entries: A) a particular facility from a set of facilities; B) a particular practitioner from a set of practitioners corresponding to the particular facility; and C) a particular treatment from a set of treatments corresponding to the particular practitioner, wherein at least one of the particular facility, the particular practitioner, or the particular treatment satisfies at least one of the subset of the plurality of criteria.

According to a further aspect, the method of the first aspect or any other aspect, wherein generating the at least one match score for each of the plurality of entries comprises: A) determining, for each of the plurality of entries; a plurality of historical ratings; and B) combining the plurality of historical ratings to generate the at least one match score.

According to a further aspect, the method of the first aspect or any other aspect, wherein combining the plurality of historical ratings comprises: A) applying a weight value to each of the plurality of historical ratings to generate a plurality of weighted ratings; and B) averaging the plurality of weighted ratings to generate the at least one match score.

According to a further aspect, the method of the first aspect or any other aspect, further comprising training and executing at least one machine learning model to estimate the weight value of each of the plurality of historical ratings.

According to a second aspect, a system, comprising: A) at least one data store; and B) at least one computing device operatively connected to the at least one data store, wherein the at least one computing device is configured to: 1) receive, from a second computing device, a request to generate a scheduling object from a particular network address, the request comprising a subset of a plurality of criteria; 2) determine a plurality of entries based on the subset of the plurality of criteria; 3) generate at least one respective match score for each of the plurality of entries; 4) generate a ranking of the plurality of entries based on each corresponding one of the at least one respective match score; 5) determine one or more top-ranked entries based on the ranking; 6) serve the one or more top-ranked entries to the particular network address; 7) receive a selection of a particular entry of the one or more top-ranked entries; 8) in response to the selection, generate the scheduling object based on the particular entry; 9) transmit the scheduling object to the particular network address; and 10) store the scheduling object at the at least one data store.

According to a further aspect, the system of the second aspect or any other aspect, wherein the plurality of criteria comprises one or more of a time interval, a location, one or more treatment preferences, and one or more product preferences.

According to a further aspect, the system of the second aspect or any other aspect, wherein the one or more treatment preferences comprise at least one treatment preference selected from the group consisting of: practitioner age, practitioner gender, treatment, treatment intensity, and treatment duration.

According to a further aspect, the system of the second aspect or any other aspect, wherein the subset of the plurality of criteria comprises a location of the second computing device.

According to a further aspect, the system of the second aspect or any other aspect, wherein the at least one computing device is configured to, in response to detecting an access event, determine a location of the second computing device, wherein at least one of the plurality of criteria is based on the location.

According to a third aspect, a non-transitory computer-readable storage medium storing a plurality of instructions executable by at least one processor, wherein the plurality of instructions, when executed by the at least one processor, cause the at least one processor to: A) receive, from a second computing device, a request to generate a scheduling object from a particular network address, the request comprising a subset of a plurality of criteria; B) determine a plurality of entries based on the subset of the plurality of criteria; C) generate at least one respective match score for each of the plurality of entries; D) generate a ranking of the plurality of entries based on each corresponding one of the at least one respective match score; E) determine one or more top-ranked entries based on the ranking; F) serve the one or more top-ranked entries to the particular network address; G) receive a selection of a particular entry of the one or more top-ranked entries; H) in response to the selection, generate the scheduling object based on the particular entry; I) transmit the scheduling object to the particular network address; and J) store the scheduling object in at least one data store.

According to a further aspect, the non-transitory computer-readable storage medium of the third aspect or any other aspect, wherein the plurality of instructions, when executed by the at least one processor, further cause the at least one processor to determine the plurality of entries by identifying, for each of the plurality of entries: A) a particular facility from a set of facilities; B) a particular practitioner from a set of practitioners corresponding to the particular facility; and C) a particular treatment from a set of treatments corresponding to the particular practitioner, wherein at least one of the particular facility, the particular practitioner, or the particular treatment satisfies at least one of the subset of the plurality of criteria.

According to a further aspect, the non-transitory computer-readable storage medium of the third aspect or any other aspect, wherein the plurality of instructions, when executed by the at least one processor, further cause the at least one processor to: A) determine, for each of the plurality of entries; a plurality of historical ratings; and B) combine the plurality of historical ratings to generate the at least one match score for each of the plurality of entries.

According to a further aspect, the non-transitory computer-readable storage medium of the third aspect or any other aspect, wherein the at least one computing device is configured to combine the plurality of historical ratings by averaging the plurality of historical ratings.

According to a further aspect, the non-transitory computer-readable storage medium of the third aspect or any other aspect, wherein the plurality of instructions, when executed by the at least one processor, further cause the at least one processor to combine the plurality of historical ratings by: A) applying a weight value to each of the plurality of historical ratings to generate a plurality of weighted ratings; and B) averaging the plurality of weighted ratings to generate the at least one match score.

According to a further aspect, the non-transitory computer-readable storage medium of the third aspect or any other aspect, wherein the plurality of instructions, when executed by the at least one processor, further cause the at least one processor to train and execute at least one machine learning model to estimate the weight value of each of the plurality of historical ratings.

These and other aspects, features, and benefits of the claimed invention(s) will become apparent from the following detailed written description of the preferred embodiments and aspects taken in conjunction with the following drawings, although variations and modifications thereto may be effected without departing from the spirit and scope of the novel concepts of the disclosure.

### BRIEF DESCRIPTION OF THE FIGURES

The accompanying drawings illustrate one or more embodiments and/or aspects of the disclosure and, together with the written description, serve to explain the principles of the disclosure. Wherever possible, the same reference numbers are used throughout the drawings to refer to the same or like elements of an embodiment.

FIG. 1 shows an exemplary event scheduling system according to one embodiment of the present disclosure.

FIG. 2 shows an exemplary event scheduling process according to one embodiment of the present disclosure.

FIG. 3 shows an exemplary scheduling process according to one embodiment of the present disclosure.

FIG. 4 shows exemplary scores associated with purposes of therapies and types of therapies according to one embodiment of the present disclosure.

FIG. 5 shows exemplary scores associated with types of therapies and body parts according to one embodiment of the present disclosure.

FIG. 6 shows an exemplary event scheduling process according to one embodiment of the present disclosure.

FIG. 7 shows an exemplary recommendation generation process according to one embodiment of the present disclosure.

FIG. 8 shows an exemplary product estimation process according to one embodiment of the present disclosure.

### DETAILED DESCRIPTION

For the purpose of promoting an understanding of the principles of the present disclosure, reference will now be made to the embodiments illustrated in the drawings and specific language will be used to describe the same. It will, nevertheless, be understood that no limitation of the scope of the disclosure is thereby intended; any alterations and further modifications of the described or illustrated embodiments, and any further applications of the principles of the disclosure as illustrated therein are contemplated as would normally occur to one skilled in the art to which the disclosure relates. All limitations of scope should be determined in accordance with and as expressed in the claims.

Whether a term is capitalized is not considered definitive or limiting of the meaning of a term. As used in this document, a capitalized term shall have the same meaning as an uncapitalized term, unless the context of the usage specifically indicates that a more restrictive meaning for the capitalized term is intended. However, the capitalization or lack thereof within the remainder of this document is not intended to be necessarily limiting unless the context clearly indicates that such limitation is intended.

### Overview

Aspects of the present disclosure generally relate to analyzing user data and matching users to facilities, personnel, and/or treatments.

Non-limiting examples of various aspects and variations of the embodiments are described herein and illustrated in the accompanying drawings.

Apparatus, methods, and systems for event scheduling are disclosed. One or more embodiments described herein generally relate to apparatus, methods, and systems for dynamically processing structured and semi-structured data, and in particular, apparatus, methods, and systems that analyze user information to match users with appropriate therapists, types of therapy and/or treatment facilities based on the structured and the semi-structured data. In some embodiments, event scheduling can be used to process, for example, stationary data, non-stationary-data, time series, linear data, non-linear data, periodic data, chaotic data, univariate data, multivariate data, and/or the like.

Described herein are event-scheduling devices that are suitable for reliable and secure matching and/or identification of appropriate therapists, types of therapy and/or treatment facilities to user information, preferences, conditions, medical history, and/or the like. One or more event-scheduling devices described herein can analyze and/or match user information with therapist expertise and facility amenities. Moreover, the one or more event-scheduling devices described herein can identify a set of time schedules that are common to the user, the therapist, and the facility. Analyzing user information with therapist and facility amenities can better utilize empty qualified facilities, can better match user needs with therapist expertise, and can further help with providing therapists with feedback and/or training for improving services or products offered. In some embodiments, a schedule can be referred to as a “docket,” or vice versa.

While the methods and apparatus are described herein as processing data from a set of files, a set of tables, a set of documents, a set of databases, and/or the like, in some instances an event-scheduling device can be used to generate the set of files, the set of tables, the set of documents, and/or the set of databases. Therefore, the event-scheduling device can be used to process and/or generate any collection or stream of data, events, and/or objects. As an example, the event-scheduling device can process and/or generate any string(s), number(s), image(s), video(s), executable file(s), dataset(s), Uniform Resource Locator (URL), global positioning system (GPS) coordinates, name(s), address(es), telephone number(s), email address(es), and/or the like. For further examples, the event-scheduling device can be used to execute or process an application programming interface (API), a function(s) of a software code(s), a webpage(s), a data file(s), a data stream(s), a model file(s), a source file(s), a script(s), a table(s) in a database system, a document-processing macro(s), an e-mail message(s), a text message(s), and/or the like.

### Exemplary Embodiments

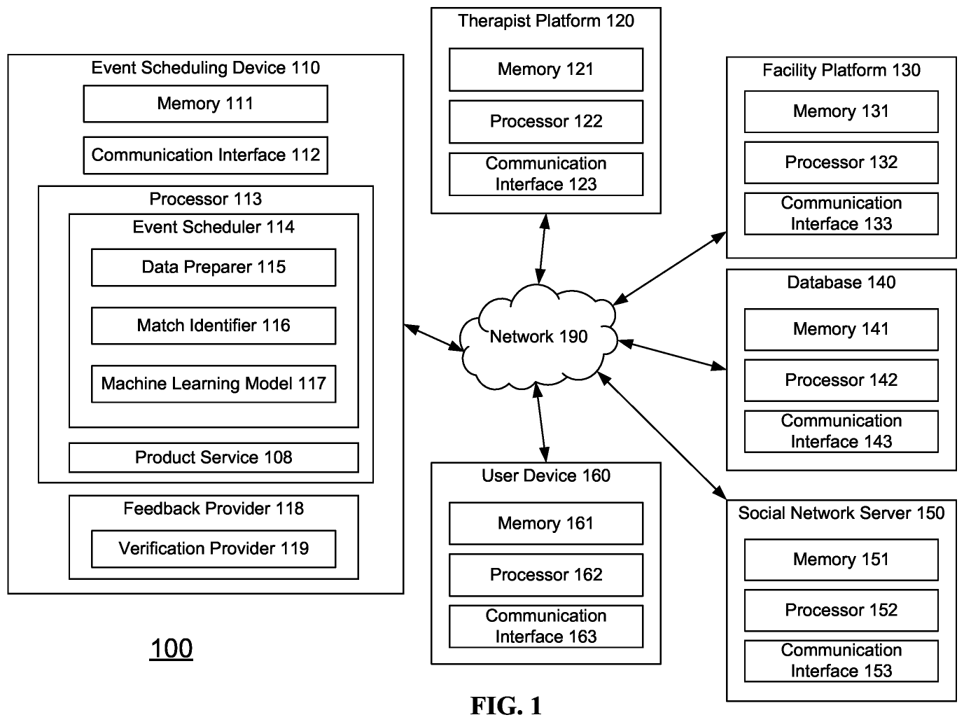

Referring now to the figures, for the purposes of example and explanation of the fundamental processes and components of the disclosed systems and processes, reference is made to FIG. 1. FIG. 1 illustrates an exemplary event scheduling system 100. As will be understood and appreciated, the exemplary event scheduling system 100 shown in FIG. 1 represents merely one approach or embodiment of the present system, and other aspects are used according to various embodiments of the present system.

FIG. 1 is a schematic description of an event scheduling system 100, according to an embodiment. The event scheduling system 100 can include one or more event-scheduling devices 110, one or more therapist platforms 120, one or more facility platforms 130, one or more databases 140, one or more social network servers 150, and one or more user devices 160. In some embodiments, one or more elements of the event scheduling system 100 are a single element that performs operations described for each component element. In one example, a therapist platform 120 and a facility platform 130 are components of a particular computing resource (e.g., a server processor, application, service, etc.) configured to execute functions with which each component is associated. In another example, the event-scheduling device 110 and the database 140 are components of a remote server environment.

The event-scheduling device 110 can receive and analyze information about a user, a set of indications of expertise of a set of therapists, a set of indications associated with types of therapies, and/or a set of indications of amenities of a set of facilities. As a result of analyzing the data, the event-scheduling device 110 can provide a set of entries, each indicating a therapist, a facility, and/or a matching score (e.g., a normalized score between 0 and 100) to the information about the user. The event-scheduling device 110 can further provide a set of available times common to the user, the set of therapists, and the set of facilities in response to a request from the user. The request may include, for example, a request to schedule a therapy appointment, a search request for a therapist and/or facility, and/or the like. The event-scheduling device 110 can be operatively coupled to the set of user devices 160, the set of therapist platforms 120, the set of facility platforms 130, a set of databases 140, and a set of social network servers 150 to transmit and/or receive data and/or analytical models via a network 190.

The event-scheduling device 110, the set of therapist platforms 120, the set of user devices 160, and/or the set of facility platforms 130 can each be a hardware-based computing device (e.g., a desktop computer, a laptop, etc.) and/or a multimedia device (e.g., a smartphone, a tablet, a wearable device, a television, an entertainment device, etc.). The event-scheduling device 110, the set of therapist platforms 120, the set of user devices 160, and/or the set of facility platforms 130 can be implemented as virtual machine elements in a cloud-computing environment. The event-scheduling device 110, the set of therapist platforms 120, the set of user devices 160, and/or the set of facility platforms 130 can include computing devices that together may include a hosted computing resource, a grid computing resource, and/or any other distributed computing arrangement. In some cases, the event-scheduling device 110, the set of therapist platforms 120, the set of user devices 160, and/or the set of facility platforms 130 can correspond to an elastic computing resource where the allotted capacity of processing, network, storage, or other computing-related re-sources may vary over time. The set of databases 140 and/or the set of social network servers 150 can each include a hardware-based computing device, a server computer, a mainframe computer, and/or the like.

The event-scheduling device 110 includes a memory 111, a communication interface 112, and a processor 113. The event-scheduling device 110 can receive data including the information about the user, the set of indications of expertise of the set of therapists, the set of indications associated with types of therapies, the set of indications of amenities in the set of facilities, a first time schedule associated with the user, a second time schedule associated with the set of therapists, and a third time schedule associated with the set of facilities, and/or the like from a set of data sources using the communication interface 112. The set of data sources can include, for example, the set of therapist platforms 120, the set of facility platforms 130, the set of databases 140, and/or the set of social network servers 150. The event-scheduling device 110 receives the data in response to a user of the event-scheduling device 110 providing a request to schedule an event (e.g., a treatment appointment, a massage appointment, a therapy appointment, and/or the like) and stores the data in the memory 111.

The memory 111 of the event-scheduling device 110 can be, for example, a memory buffer, a random access memory (RAM), a read-only memory (ROM), a hard drive, a flash drive, a secure digital (SD) memory card, a compact disk (CD), an external hard drive, an erasable programmable read-only memory (EPROM), an embedded multi-time programmable (MTP) memory, an embedded multi-media card (eMMC), a universal flash storage (UFS) device, and/or the like. The memory 111 can store, for example, one or more software modules and/or code that includes instructions to cause the processor 113 to perform one or more processes and/or functions (e.g., an event scheduler 114, a data preparer 115, a match identifier 116, a machine learning model 117, a feedback provider 118, and/or a verification provider 119).

The memory 111 can store a set of files associated with (e.g., generated by executing) the event scheduler 114, the data preparer 115, the match identifier 116, the machine learning model 117, the feedback provider 118, and/or the verification provider 119. The set of files can include data generated by the event scheduler 114, the data preparer 115, the match identifier 116, the machine learning model 117, the feedback provider 118, and/or the verification provider 119 during the operation of the event-scheduling device 110. For example, the set of files can include temporary variables, return memory addresses, variables, a table of the feedback provider 118 (e.g., a set of evaluation scores), a graph associated with the machine learning model 117 (e.g., a set of arithmetic operations or a representation of the set of arithmetic operations used by the machine learning model 117), and/or the like, generated during the operation of the event-scheduling device 110.

The communication interface 112 of the event-scheduling device 110 can be a hardware component and/or a software component (executed by hardware) of the event-scheduling device 110 to facilitate data communication between the event scheduling device 110 and external devices (e.g., the set of therapist platforms 120, the set of facility platforms 130, the set of databases 140, and the set of social network servers 150, etc.) and/or internal components of the event-scheduling device 110 (e.g., the processor 113, the memory 111, etc.). The communication interface 112 can include, for example, a network interface card (NIC), a Wi-Fi™ module, a Bluetooth® module, an optical communication module, and/or any other suitable wired and/or wireless communication interface. The communication interface 112 can be configured to connect the event-scheduling device 110 to the network 190. In some instances, the communication interface 112 can facilitate receiving or transmitting the data via the network 190. More specifically, in some implementations, the communication interface 112 can facilitate receiving or transmitting data through the network 190 from or to the set of therapist platforms 120, the set of facility platforms 130, the set of databases 140, and/or the set of social network servers 150, each communicatively coupled to the event-scheduling device 110 via the network 190. In some instances, data received via the communication interface 112 can be processed by the processor 113 or stored in the memory 111, as described in further detail herein.

In some implementations, the communication interface 112 can facilitate an authenticated communication, via the network 190, between the event-scheduling device 110 and the set of therapist platforms 120, the set of facility platforms 130, the set of databases 140, the set of user devices 160, and/or the set of social network servers 150. In some implementations, the authenticated communication may be established by exchanging a set of encoded signals between the event-scheduling device 110 and the set of therapist platforms 120, the set of facility platforms 130, the set of databases 140, and/or the set of social network servers 150.

The processor 113 can be, for example, a hardware-based integrated circuit (IC) or any other suitable processing device configured to run or execute a set of instructions or a set of codes. For example, the processor 113 can include a general-purpose processor, a central processing unit (CPU), an accelerated processing unit (APU), an application-specific integrated circuit (ASIC), a field-programmable gate array (FPGA), a programmable logic array (PLA), a complex programmable logic device (CPLD), a programmable logic controller (PLC), a graphics processing unit (GPU), a neural network processor (NNP), and/or the like. In some instances, the processor 113 can be operatively coupled to the memory 111 through a system bus (for example, address bus, data bus, and/or control bus, not shown).

The processor 113 executes the event scheduler 114 and the feedback provider 118. Each of the event scheduler 114 and/or the feedback provider 118 can include software stored in the memory 111 and executed by the processor 113. For example, a code to cause the event scheduler 114 to generate an entry and/or a schedule (e.g., indicating a therapist, a facility, a set of days, and/or a set of times) can be stored in the memory 111 and executed by the processor 113. Alternatively, each of the event scheduler 114 and/or the feedback provider 118 can be a hardware-based device. For example, a process to cause the event scheduler 114 to generate the entry and/or the schedule can be implemented on an individual integrated circuit chip.

The event scheduler 114 includes a data preparer 115 and a match identifier 116. Each of the data preparer 115 and/or the match identifier 116 can include software stored in the memory 111 and executed by the processor 113. Alternatively, each of the data preparer 115 and/or the match identifier 116 can be a hardware-based device. The event scheduler 114 can receive the data including the information about the user, the set of indications of expertise of the set of therapists, the set of indications associated with types of therapies, the set of indications of amenities in the set of facilities, the first time schedule associated with the user, the second time schedule associated with the set of therapists, and the third time schedule associated with the set of facilities, and/or the like from the memory. In some implementations, the communication interface 112 can facilitate the event scheduler 114 to receive the data directly from the set of data sources.

The data preparer 115 can process and modify the data to better analyze the data. In some instances, the data preparer 115 can modify each data to a normalized and/or standardized format for more accurate and/or reliable analysis, as describe in further detail herein. In some instances, the data preparer 115 can partition the data in such a way to process the data faster and better use storage and/or computing resources of the event-scheduling device 110, the set of therapist platforms 120, the set of facility platforms 130, the set of database platforms 140, and/or the set of social network servers 150. For example, the processor 113 of the event-scheduling device 110 can include 8 processing cores with same clock rate. The data preparer 115 can divide the data to multiple partitions to process them in parallel, such as in 2 partitions, 4 partitions, 6 partitions, 8 partitions, 12 partitions, or 16 partitions, among others. In some implementations, the data preparer 115 can partition the data for more reliable or more secure data transfer via the network 190 between the event-scheduling device 110 and the set of therapist platforms 120, the set of facility platforms 130, the set of database platforms 140, and/or the set of social network servers 150.

In some implementations, the data preparer 115 iteratively receives batches of data from the set of data sources (e.g., the set of therapist platforms 120, the set of user devices 160, the set of facility platforms 130, the set of databases 140, and/or the set of social network servers 150) and/or the memory 111. Each batch of data can include a subset of information about the user, a subset of indications of expertise of a subset of therapists, a subset of indications associated with types/purpose of therapies, a subset of indications of amenities in a subset of facilities, and/or a subset of schedules for the user, subset of therapists and/or the subset of facilities.

In some implementations, the data preparer 115 can extract features from the data. For example, the data preparer 115 can extract and organize strings, numbers, names, durations, platform types, file types, web browsers, operating systems, and/or the like in the data. The data preparer 115 can be configured, for example, to represent the extracted features as a set of feature vectors and/or other suitable format.

In some implementations, the data preparer 115 can perform a set of normalization steps. The set of normalization steps can include normalizing each feature vector to a common scale and/or transforming data and/or feature vectors to a common format to improve analyzing the data and/or feature vectors in the event-scheduling device 110. For example, the data preparer 115 can generate feature vectors and/or data with predetermined length and/or numerical range. The processor 113 can then present the feature vectors and/or data with predetermined length and/or numerical range to the match identifier 116.

The match identifier 116 can be configured to accept the data and/or the feature vectors from the data preparer 115 to detect a set of entries for the user of the event-scheduling device 110 as an output. In some implementations, the match identifier 116 can receive the data directly from the memory 111 and/or the set of data sources and without any processing of the data by the data preparer 115. The match identifier 116 can be configured to detect a set of entries, each entry indicating a therapist from the set of therapists, a facility from the set of facilities, and a calculated matching score in response to the request. In some implementations, the calculated matching score can be a representation of a likelihood that the therapist has expertise/skills that can help a particular need of the user and/or the facility has amenities that the user may use and/or amenities that the therapist may use to help the user. The match identifier 116 can further detect a set of free time schedules common to the user, the therapist, and the facility in response to the request. In some implementations, the match identifier can calculate the calculated matching score (e.g., a percentage value, a normalized number between −1 and 1, a Boolean indicator (e.g., yes or no), a rank) based on the information about the user (e.g., personal preferences, medical history, user profile), an indication of expertise of the therapist (e.g., a set of scores of the therapist for a set of conditions across a set of therapy types) and/or an indication of amenities of the facility.

For example, the personal preferences can include an indication of preference for a male therapist, an indication of preference for a female therapist, an indication of preference for a young therapist, an indication of preference for an old therapist, and/or the like. The personal preferences can include an indication of preference for an expertise in Shiatsu massage, an indication of preference for a certified massage therapist in pre-natal massage, an indication of preference for expertise of therapy for a body part, an indication of preference for expertise in shoulder therapy, and indication of preference for expertise in leg therapy, and/or the like. The personal preferences can include an indication of preference for a medical center, an indication of preference for a hospital, an indication of preference for a gym, an indication of preference for a spa, an indication of preference for a resort, and/or the like.

In some instances, the user profile of the user may include biographic information (e.g., name, age, sex, ethnicity, etc.), a set of past personal preferences (e.g., Shiatsu massage), a set of current personal preference (e.g., pre-natal massage), medical history (shoulder pain, broken rib, muscular dystrophy, etc.), and/or the like. The user profile can be stored in the event-scheduling device 110, the set of therapist platforms 120, the set of user devices 160, the set of facility platforms 130, the set of databases 140, and/or the set of social network server 150. In some implementations, the user profile can be initially generated by an operator of the event-scheduling device 110 to include information about the user such as, for example, biographic information, medical conditions, and/or the like. The user profile can then be updated and/or modified by the user (via the user device 160) to include updated information about the user that may in part or in full be different from the information about the user. In some implementations, the event-scheduling device 110 can transmit a signal using the communication interface 112 and via the network 190 to update the user profile stored in the set of therapist platforms 120, the set of user devices 160, the set of facility platforms 130, the set of databases 140, and/or the set of social network servers 150.

In some implementations, the match identifier 116 can optionally include a machine learning model 117 that can be trained to learn an underlying pattern of matchings between historical data and historical entries, each matching including a matching score. In some implementations, the machine learning model 117 can include, for example, an artificial neural network having a set of layers, each having a set of parameters including a set of weights, a set of biases, and/or a set of activation functions. The set of parameters including the set of weights, the set of biases, and the set of activation functions can collectively perform a set of arithmetic operations to correlate the historical data to the historical entries while attempting to optimize/improve a cost function. Once trained, the machine learning model can receive data including information about a user, indications of expertise of therapists, and/or indications of amenities of facilities and generate entries with a matching score meeting preset criteria. In some implementations, the machine learning model 117 can include one or more convolutional neural networks, Bayesian learning models, recurrent neural networks, Gaussian mixture models, unsupervised machine learning models, and/or the like.

The feedback provider 118 can be configured to receive the set of entries from the match identifier 116 and/or the set of data and/or feature vectors from the data preparer 115, the memory 111, and/or the set of databases to audit the set of entries. The set of entries can be displayed to the user of a user device 160 and/or the event-scheduling device 110 to be evaluated (e.g., approved, disapproved, scored, ranked, and/or the like). In some instances, the user can evaluate each entry via a feedback application programming interface (API). For example, a user having insomnia can evaluate, via the feedback API, an entry suggesting a therapist that has a Reiki score of 5 out of 5 and works in the same city/county/neighborhood as the user. In some implementation, evaluations of the user can be stored in the memory 111 and/or the set of databases as user information/user preferences. For example, a user celebrating a first occasion at a first time may evaluate a therapist and a facility as rank number 1. The rank number, the therapist, the facility and an indicator of the first occasion can be stored in a database from the set of databases 140. Similarly stated, feedback provider 118 can be used to update the user's preferences. In one example, a set of past evaluations provided by the user can be associated with the user's profile and stored in the event-scheduling device 110, the set of therapist platforms 120, the set of user devices 160 (e.g., in the user device associated with that user), the set of facility platforms 130, the set of databases 140, and/or the set of social network servers 150. When the user submits a request to schedule another event for a second occasion at a second time after the first time (e.g., via the user device associated with that user), the event-scheduling device 110 can include an entry suggesting the same therapist and the same facility as rank number one to the user. In some implementations, the evaluations can be used as additional/alternative training data to train the machine learning model 117.

The feedback provider 118 can optionally include a verification provider 119 that receives entries generated by the machine learning model 117 upon a user request from a user device 160 for scheduling an event (e.g., a therapy session). The user device 160 is operatively coupled to the event-scheduling device 110. Each entry can include information about the user of the user device 160, indications of expertise of therapists, and/or indications of amenities of facilities and a set of entries generated by the machine learning model 117. Each entry can be further associated with a matching score also generated by the machine learning model 117. In some implementations, the verification provider 119 can transmit, via the network 190, an inquiry to the user via a graphical user interface (GUI) of the user device 160 to input a set of verification scores. The set of verification scores can be compared to the matching score to generate a set of deviations. The set of verification scores, the set of matching scores, and/or the set of deviations can be stored in the memory 111 and/or be used to further train the machine learning model 117 and/or update the user's profile.

The processor 113 can include a product service 108. As used herein, product can generally refer to any purchasable good, service, or other resource (e.g., resource may be used interchangeable with product). The product service 108 can analyze product consumption data and scheduling data to generate various determinations, such as, for example, future product consumption levels, future product amount requirements, and product ordering intervals. The product service 108 can analyze historical product consumption data and generate one or more consumption trends based on the analysis. The consumption trend can refer to a model or equation that takes, as input, historical product consumption data and a current appointment schedule, and generates, as output, an estimate for an amount of product that may be required to satisfy the current appointment schedule. In some embodiments, a consumption trend is referred to as a consumption rate. The product service 108 can estimate a date and time at which an entity may exhaust its supply of a particular product. The product service 108 can estimate a date and time by which an entity should order an estimated amount of a particular product to ensure the particular product is available for each appointment of the current schedule. In some embodiments, the product service 108 can automatically place an order for a fixed amount of the particular product. As used herein, the term automatic can refer to being performed by a computing device with limited or no human interaction being necessary.

The product service 108 can communicate with one or more supplier systems and/or analyze historical supplier data to determine an ordering interval for one or more products. The ordering interval can refer to a time and date by which a particular product amount should be ordered from a supplier to ensure that the particular product is available for each appointment of the current schedule. For example, the product service 108 queries a supplier system to determine a current stock of a particular product and an estimated delivery interval for delivering a particular amount of the particular product to an entity (e.g., to a facility associated therewith or another suitable location).

The network 190 can be a digital telecommunication network of servers and/or computing devices. The servers and/or computing device on the network can be connected via a set of wired and/or wireless communication networks (not shown) to share resources such as, for example, data storage and/or computing power. The wired and/or wireless communication networks between servers and/or computing devices of the network 190 can include a set of communication channels, for example, a radio frequency (RF) communication channel(s), a fiber optic commination channel(s), an electronic communication channel(s), a satellite communication channel(s), and/or the like. The network 190 can be, for example, the Internet, an intranet, a local area network (LAN), a wide area network (WAN), a metropolitan area network (MAN), a worldwide interoperability for microwave access network (WiMAX®), a virtual network, any other suitable communication system and/or a combination of such networks.

The set of therapist platforms 120 can include computing devices operatively coupled to the event-scheduling device 110. Specifically, each therapist platform 120 from the set of therapist platforms 120 is a computing device that can transmit and/or receive data and/or analytical models to the event-scheduling device 110. In some instances, each therapist platform 120 can be associated with a different therapist (e.g., can be a computing device of that therapist). In some implementations, the set of therapist platforms 120 can be configured to transmit, via the network 190, the set of indications of expertise of the set of therapists to the event-scheduling device 110. Specifically, the therapist platform 120 of a given therapist can transmit, via the network 190, one or more indications of expertise of the therapist associated with that therapist platform, a location associated with that therapist, preferences associated with that therapist, and/or any other information associated with that therapist. Each therapist platform 120 from the set of therapist platforms can include, for example, a personal computer(s), laptop(s), smartphone(s), and/or so forth, each including a memory 121, a communication interface 123 and a processor 122. The processor 122 can include a hardware-based integrated circuit (IC) or any other suitable processing device configured to run and/or execute a set of instructions or code. The memory 121 can include a hardware-based charge storage electronic device or any other suitable data storage medium configured to store data. In some implementations, a therapist using a therapist platform 120 can provide a set of indications of therapist expertise based on previous feedback from clients, therapy certificates, therapy trainings, and/or the like to be stored in the therapist platform 120. The therapist platform 120 can transmit the set of indications of therapist expertise to the event-scheduling device 110, the set of databases 140, and/or the set of social network servers 150 via the network 190.

In some instances, each indication of expertise can include an expertise string and/or an expertise score. The expertise string can include, for example, stretching, deep tissue, neuromuscular therapy, sports massage, trigger point massage, Rolfing, reflexology, Swedish, pre-natal, table Thai, Shiatsu, Reiki, cranial sacral, and/or the like. The expertise score can include, for example, a score between 0 to 5, a score between 0 to 10, a normalized score between 0 to 1, a percentage value, a Boolean number, an/or the like. In some instances, each expertise string can have a unique number for each of a preference, a medical condition, and/or an occasion of the user of the user device 160, past clients, or current patients. The preference, the medical conditions, and/or the occasion of the user can include, for example, relaxation, vacation, celebration, pain in shoulder, pain in leg, tension, stress, overall wellbeing, insomnia, migraines, pregnancy, light pressure, medium pressure, firm pressure, deep pressure, and/or the like.

The set of facility platforms 130 can include computing devices operatively coupled to the event-scheduling device 110. Specifically, each facility platform from the set of facility platforms 130 is a computing device that can transmit and/or receive data and/or analytical models to and/or from the event-scheduling device 110 and/or the set of therapist platforms 120. In some instances, each facility platform 120 can be associated with a different facility (e.g., can be a computing device of that facility). In some implementations, each facility platform 130 can be configured to transmit, via the network 190, the set of indications of amenities and/or capabilities of an associated facility to the event-scheduling device 110. Each facility platform from the set of facility platforms 130 can include, for example, a personal computer(s), a laptop(s), a smartphone(s), or so forth, each including a memory 131, a communication interface 133 and a processor 132. The processor 132 can include a hardware-based integrated circuit (IC) or any other suitable processing device configured to run and/or execute a set of instructions or code. The memory 131 can include a hardware-based charge storage electronic device or any other suitable data storage medium configured to store data. In some instances, an indication of an amenity can include an amenity string and/or an amenity availability schedule. The amenity string can include, for example, pool, Jacuzzi bath, table, gym, and/or the like.

The set of databases 140 can include computing device mediums particularly for data storage purpose and can include, for example, a network of electronic memories, a network of magnetic memories, a server(s), a blade server(s), a storage area network(s), a network-attached storage(s), deep learning computing servers, deep learning storage servers, and/or the like. Each database 140 can include a memory 141, a communication interface 143, and a processor 142. The memory 141 can store the data, the processor 142 can analyze the data, and the communication interface 143 can receive/transmit the data from/to the event-scheduling device 110 via the network 190. In some implementations, the set of databases 140 can include and/or execute a cloud-based service such as, for example, a software as a service (SaaS), a platform as a service (PaaS), an infrastructure as a service (IaaS), and/or the like.

The set of social network servers 150 can include servers and/or computing devices associated with social media services, such as WeChat®, Reddit®, Facebook®, YouTube®, LinkedIn®, Pinterest®, Twitter®, and/or the like. Specifically, each social network server from the set of social network servers 150 is a computing device that can send and receive data. Specifically, each social network server 150 can transmit and/or receive data, API requests, and/or analytical models to and/or from the event-scheduling device 110, the set of user devices 160, the set of therapist platforms 120, the set of facility platforms 130, and/or the set of databases 140. In some instances, each social network server 150 can include information about a user (e.g., computing device of that user), a therapist (e.g., computing device of that therapist), and/or a facility (e.g., a computing device of that facility). The information about the user, the therapist, and/or the facility can include a social media profile(s), past interaction with an online content(s) (e.g., an advertisement, a photo, a web link, etc.), a graph(s) of social connections, an online review(s), and/or the like, associated to the user, the therapist, and/or the facility. In some implementations, such information can be retrieved and used when calculating a matching score and/or a rank score, as described herein.

Each social network server 150 includes a memory 151, a processor 152, and/or a communication interface 153. The processor 152 can be, for example, a hardware-based integrated circuit (IC) or any other suitable processing device configured to run and/or execute a set of instructions or code. For example, the processor 152 can be a general purpose processor, a central processing unit (CPU), an accelerated processing unit (APU), an application specific integrated circuit (ASIC), a field programmable gate array (FPGA), a programmable logic array (PLA), a complex programmable logic device (CPLD), a programmable logic controller (PLC) and/or the like. The processor 152 can be operatively coupled to the memory 151 through a system bus (for example, address bus, data bus and/or control bus). The memory 151 can be, for example, random access memory (RAM), memory buffers, hard drives, databases, erasable programmable read only memory (EPROMs), electrically erasable programmable read only memory (EEPROMs), read only memory (ROM), cloud storage, and/or the like. The set of social network servers 150 can operatively couple to the event-scheduling device 110 to transmit/receive, via the network 190, the information about the user, the therapist, and/or the facility included in the set of social network servers 150 to/from the event-scheduling device 110. In some implementations, the social network server 150 can transmit/receive the information about the user, the therapist, and/or the facility based on a connectivity to and/or authentication from the user device 160, the facility platform 130, and/or the therapist platform. In some implementations, such information can be retrieved and used when calculating a matching score and/or a rank score, as described herein.

Each user device from the set of user devices 160, can include a computing device(s) (e.g., a laptop(s), a phone(s), a smart watch(es), a smart mirror(s), a personal assistant, and/or the like) operatively coupled to the event-scheduling device 110. Each user device from the set of user devices 160 can transmit and/or receive data (e.g., the information about a user of that user device, the user preferences, the user's medical history, the set of entries, the user's location, a request to schedule a therapy appointment, a search request for a therapist and/or facility, and/or the like) to/from the event-scheduling device 110. Each user device 160 includes a memory 131, a communication interface 133 and a processor 132. The processor 132 can include a hardware-based integrated circuit (IC) or any other suitable processing device configured to run and/or execute a set of instructions or code. The memory 131 can include a hardware-based charge storage electronic device or any other suitable data storage medium configured to store data. In some instances, the user device 160 can include and/or be operatively coupled to a user interface (e.g., a keyboard, a mouse, a monitor, a digital screen, and/or the like). The user interface can facilitate presentation of the set of entries to the user and receiving commands from the user. In some implementation, the user device can include an app (e.g., an application software) that handles (e.g., display the set of entries, receive a score) interactions of the user with the user device 160 and the interactions of the user device 160 with the event-scheduling device 110.

In use, in response to a user's request from a user device (e.g., a request to schedule a therapy appointment, a search request for a therapist and/or facility, etc.), the event-scheduling device 110 can receive data including the information about the user and the first time schedule associated with the user from a user device 160 (e.g., in response to the user of the user device 160 selecting to schedule an appointment for therapy providing information biographic information, health conditions, and/or preferences), the set of databases 140, and/or the set of social network servers 150. The event-scheduling device 110 can receive data including the set of indications of expertise of the set of therapists and the second time schedule associated with the set of therapists from the set of therapist platforms 120, the set of databases 140, and/or the set of social network servers 150. The event-scheduling device 110 can receive data including the set of indications of the set of amenities and/or capabilities of the set of facilities and the third time schedule associated with the set of facilities from the set of facility platforms 130, the set of databases 140, and/or the set of social network servers 150. In some instances, the event-scheduling device 110 can access a first calendar in the user device 160, a second set of calendars in the set of therapist platforms 120, and/or a third set of calendars in the set of facility platforms 130 to access the first time schedule associated with the user, the second time schedule associated with the therapists and/or the third time schedule associated with the facilities, respectively. The event-scheduling device 110 can use the data to generate a set of entries. Each entry can include matching the information about the user with a therapist from the set of therapists, a facility from the set of facilities, and a matching score calculated by the event scheduler 114. The event-scheduling device 110 can further use the first time schedule, the second time schedule, and the third time schedule to detect a set of free time schedules common to the user, the therapist, and the facility.

In some implementations, the event-scheduling device 110 can analyze the information about the user and the first time schedule associated with the user, the set of indications of expertise of the set of therapists and the second time schedule associated with the set of therapists, the set of indications of the set of amenities of the set of facilities, and the third time schedule associated with the set of facilities. As such, the event-scheduling device 110 can account for the availability common to the user, the therapist, and the facility when calculating the matching score for each entry.

The event-scheduling device 110 can then present the set of entries to the user in a way that the user can interact with the set of entries (e.g., see more details in an expanded view, select an entry, remove an entry, rate an entry, add an entry to favorites, disapprove an entry, and/or the like). In some implementations, the set of entries can be ranked based on their matching scores. The user can then select (e.g., via an app on the user device 160) an entry to schedule an appointment with the therapist and the facility.

In some instances, the user may provide (e.g., via an app/application on the user device 160) feedback (e.g., a set of numerical ratings, a set of ranking, a set of verbal feedback recorded and/or processed by the event-scheduling device 110, and/or the like) to the event-scheduling device 110. In response to receiving the feedback from the user, the event-scheduling device 110 can calculate a rank score for the therapist and/or the facility. The rank score is a comparative and/or hierarchical numerical value that can be assigned and/or associated with the therapist and/or the facility (e.g., a rating, rank among similar therapists and/or facilities, etc.).

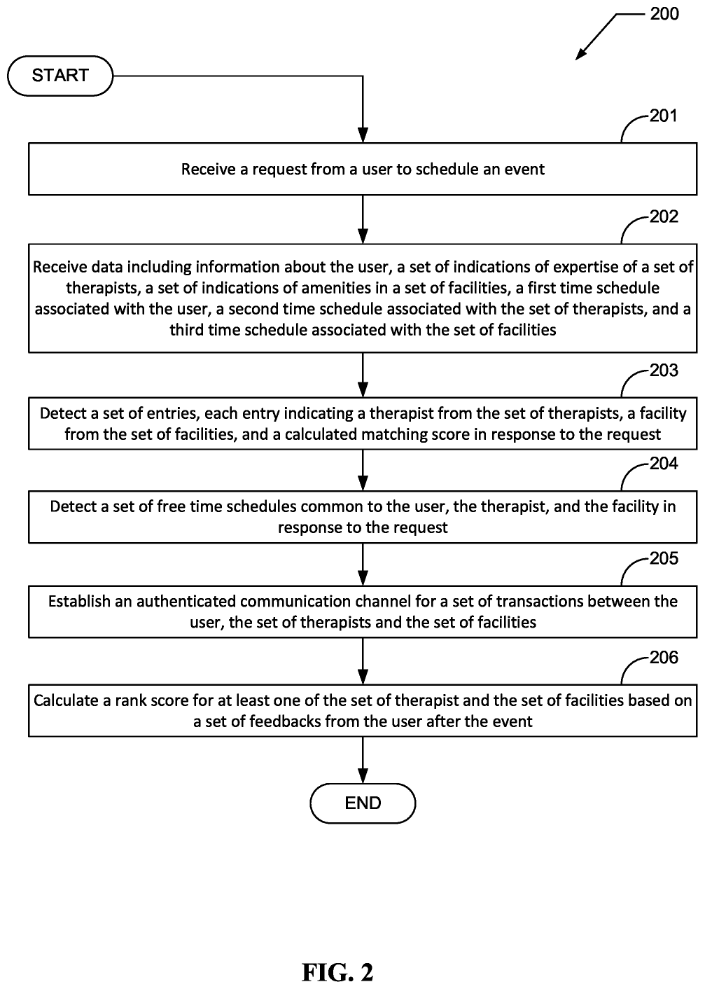

FIG. 2 is a flowchart illustrating an event scheduling process 200, according to an embodiment. As will be understood by one having ordinary skill in the art, the steps and processes shown in FIG. 2 (and those of all other flowcharts and sequence diagrams shown and described herein) may operate concurrently and continuously, are generally asynchronous and independent, and are not necessarily performed in the order shown. In some embodiments, an event-scheduling device (such as the event-scheduling device 101 as shown and described with respect to FIG. 1) can perform the process 200.

At step 201, the process 200 includes receiving a request from a user. For example, the event-scheduling device 110 can be configured to receive a request from a user (e.g., of the event-scheduling device, or a device operatively coupled to the event-scheduling device, and/or the like) to schedule an event. The request can include a set of preferences of the user and a first time schedule associated with the user.

At step 202, the process 200 includes receiving data associated with the user. For example, the event-scheduling device can be configured to receive data including the information about the user, a set of indications of expertise of a set of therapists, a set of indications of amenities and/or capabilities in a set of facilities, a first time schedule associated with the user, a second time schedule associated with the set of therapists, and a third time schedule associated with the set of facilities. The information can include, for example, name, age, sex, height, ethnicity, medical conditions, medications, past chiropractic treatments, past interaction with social networks, Internet history, the set of preferences of the user, and/or the like. In some instances, the first time schedule, the second time schedule, and/or the third time schedule can include, for example, a set of 60-minute time slots, a set of 90 minute time slot, and/or the like for duration of a day(s), a week(s), a month(s), and/or the like.

At step 203, the process 200 includes detecting a set of entries. For example, the event-scheduling device can be configured to detect a set of entries, each entry indicating a therapist from the set of therapists, a facility from the set of facilities, and a calculated matching score in response to the request. The calculated matching score can be a representation of a likelihood that the therapist has expertise/skills to match the request of the user and/or the information about the user, and/or the facility has amenities that the user or the therapist may use.

At step 204, the process 200 includes detecting a set of free time schedules. For example, the event-scheduling device can be configured to detect a set of free time schedules common to the user, the therapist, and the facility in response to the request.

At step 205, the process 200 includes establishing an authenticated communication channel for facilitating communication and/or transactions between a computing device of the user, the set of therapists, and the set of facilities. For example, the event-scheduling device can be configured to optionally establish an authenticated communication channel for a set of transactions between the user, the set of therapists and the set of faculties. In some instances, the authenticated communication may be established by exchanging a set of encoded signals between the event-scheduling device and a set of therapist platforms of the therapist, and a set of facility platforms of the facilities, a set of databases, and/or a set of social network servers. In some embodiments, the set of transactions may involve a set of financial transaction, a set of private messages, a set of digital assets, a set of cryptocurrencies, and/or the like. In some implementations, the event-scheduling device can be configured to rank the set of entries based on at least one of the calculated matching score and the set of free time schedules. The event-scheduling device can be further configured to prompt the user (e.g., via a GUI of a user device) to select at least one entry from the set of entries to schedule an appointment with a therapist and at a facility. In some implementations, the user can provide feedback (e.g., after the scheduled appointment).

At step 206, process includes calculating one or more rank scores for ranking and/or classifying the set of therapists and/or the set of facilities. For example, the event-scheduling device can be configured to calculate a rank score for at least one of the set of therapists and the set of facilities based on feedback from the user after the event. In some instances, the rank score can be further take into account, for example, feedback from other users (e.g., past users, current users), therapist credentials, facility rating on a social network server, and/or the like.

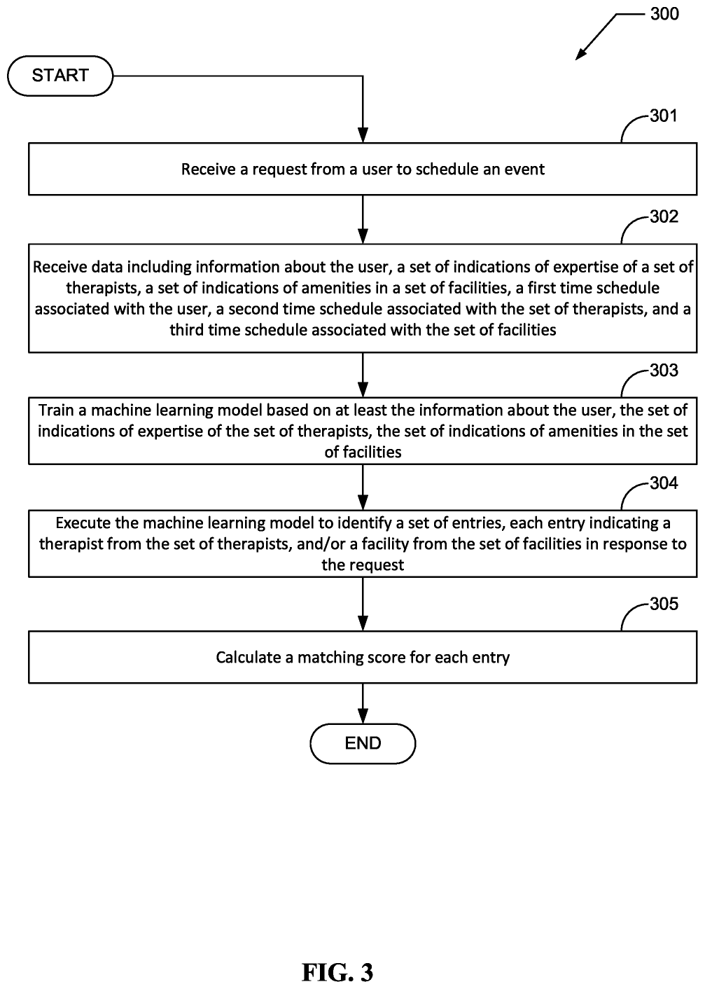

FIG. 3 is a flowchart illustrating an event scheduling process 300, according to various embodiments of the present disclosure. In some implementations, an event-scheduling device (such as the event-scheduling device 101 as shown and described with respect to FIG. 1) can be used to perform the process 300.

At step 301, the process 300 includes receiving an event scheduling request from a user. For example, the event-scheduling device can be configured to receive a request from a user to schedule an event.

At step 302, the process 300 includes receiving data associated with the user. For example, the event-scheduling device can be configured to receive data including information about the user, a set of indications of expertise of a set of therapists, a set of indications of amenities in a set of facilities, a first time schedule associated with the user, a second time schedule associated with the set of therapists, and a third time schedule associated with the set of facilities.

At step 303, the process 300 includes training one or more machine learning models to identify, in response to the request, one or more entries, each entry including a therapist from the set of therapists and/or a facility from the set of facilities. For example, the event-scheduling device can be configured to train a machine learning model based on at least the information about the user, the set of indications of expertise of the set of therapists, the set of indications of amenities in the set of facilities.

At step 304, the process 300 includes executing the machine learning model to generate, as output, at least one entry corresponding to the request. For example, the event-scheduling device can be configured to execute the machine learning model to identify a set of entries, each entry indicating a therapist from the set of therapists, and/or a facility from the set of facilities in response to the request.

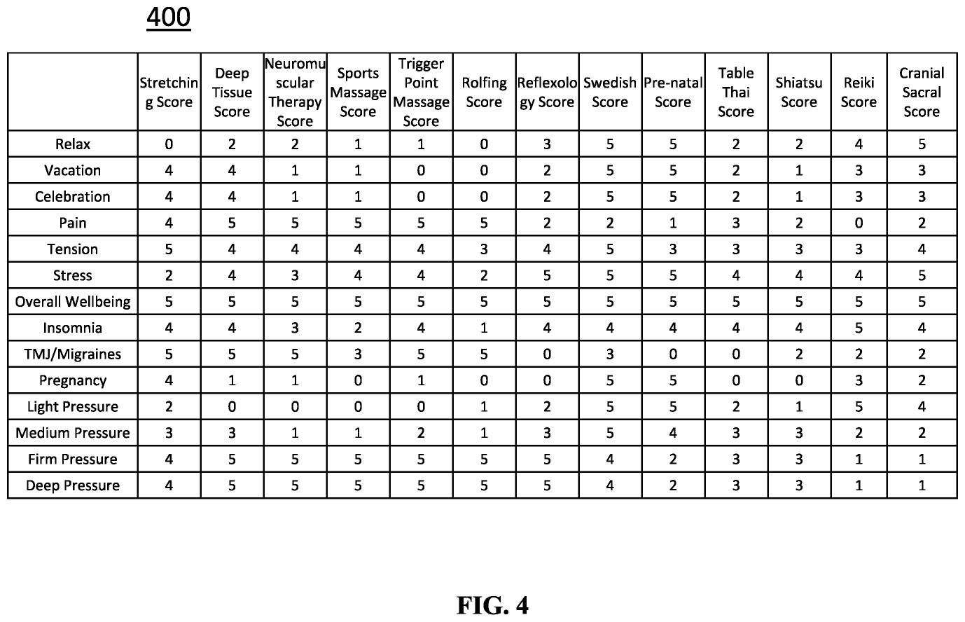

At step 305, the process 300 includes calculating a matching score for each entry. For example, the event-scheduling device can be configured to calculate a matching score for each entry. In some instances, the event-scheduling device can be configured to rank the set of entries based on at least one of the matching score and a common schedule between the user, the set of therapists, and/or the set of facilities. The event-scheduling device can be further configured to prompt the user (e.g., via a GUI of a user device) to select at least one entry from the set of entries to schedule an appointment. FIG. 4 is a table 400 illustrating scores associated with purposes of therapies and types of therapies, according to an embodiment.

As shown in FIG. 4, a first score table can be associated with purposes of therapies and types of therapies. Each score in the first score table can serve as a measure of effectiveness of a type of therapy for a purpose of therapy. In some instances, the first score table can be filled by generally accepted scores for effectiveness of each pair from type of therapies and purposes of therapies. In some implementations, the generally accepted scores can be updated on a regular basis (e.g., daily, weekly, monthly, annually, etc.) or be updated once a set of updated scores are made available (e.g., by an association, a community, etc.). The purposes of therapies can include occasions and/or conditions including, for example, relaxation, vacation, celebration, pain, tension, stress, overall wellbeing, insomnia, migraines, pregnancy, light pressure, medium pressure, firm pressure, deep pressure, and/or the like. The types of therapies can include, for example, stretching, deep tissue, neuromuscular therapy, sports massage, trigger point massage, Rolfing, reflexology, Swedish, pre-natal, table Thai, Shiatsu, Reiki, cranial sacral, and/or the like.

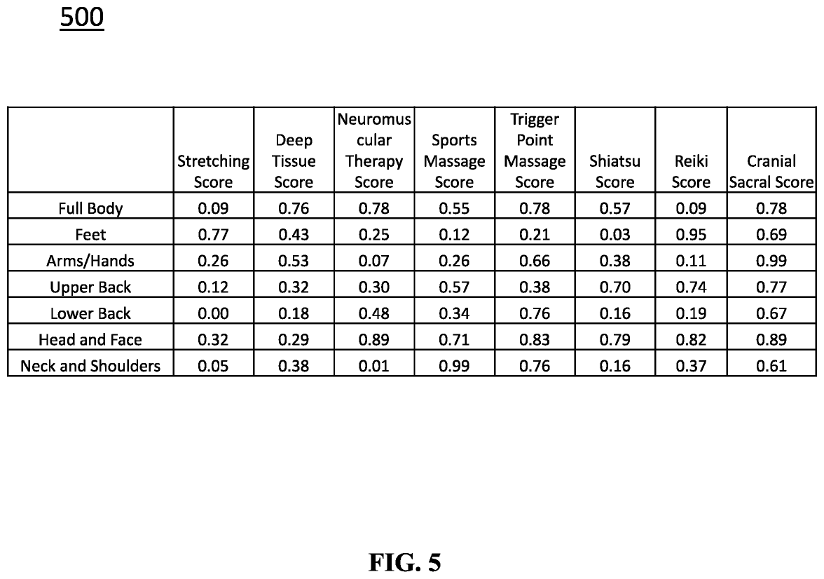

FIG. 5 is a table 500 illustrating scores associated with types of therapies and body parts helped by the therapies, according to an embodiment. As shown in FIG. 5, a second score table can be associated with types of therapies and body parts helped by the therapies. Each score in the second score table can serve as a measure of effectiveness of a type of therapy for a body part. In some instances, the second score table can be filled by generally accepted scores for effectiveness of each pair from type of therapies and body parts. In some implementations, the generally accepted scores can be updated on a regular basis (e.g., daily, weekly, monthly, annually, etc.) or be updated once a set of updated scores are made available (e.g., by an association). The body parts helped by the therapies can include, for example, neck, shoulders, head, face, lower back, upper back, arms, hands, feet, full body, and/or the like.

In some embodiments, a third score table (not shown) can be associated with therapists and types of therapies. In some instances, each score in the third score table can be provided by a previous client(s) (e.g., via the user device 160 described with respect to FIG. 1) for a therapy session performed by a therapist(s). In some instances, each score in the score table can be provided by the therapist(s) with whom that score table is associated, by peer therapists (e.g., the set of therapist platforms 120 described with respect to FIG. 1), and/or by an organization that test and rate therapists. In some instances, each score in the score table can be calculated based on a formula (e.g., a preset formula). For example, a first score normalized to 1 representing average scores provided by past clients can be added to a second score normalized to 1 and provided by the therapist, and further added by a factor (e.g., multiplied by 3) of a third score normalized to 1 and provided by the organization. In some instances, the previous client(s) can provide the score on a user device (similar to the user device 160 as shown and described with respect to FIG. 1).

An event-scheduling device can use an algorithm to provide personalized scores for a user and provide entries/recommendations to the user based on scores from the first score table, the second score table, and/or the third score table. The algorithm can include a set of arithmetic procedures and/or logical procedures to calculate the personalized scores and provide the entries/recommendations. In some implementations, a user can provide (e.g., via an application on a user device and/or as previously stored in a user profile) to an event-scheduling device an indication of an occasion, a type of treatment, a body part for therapy, and/or any other details and/or preferences regarding a treatment appointment. Using the scores from the first table, the second table, and/or the third table, a personalized score and/or recommendation can be provided to that user. For example, a user can provide, via the application on the user device, a request for an overall wellbeing purpose of therapy for a lower back body part. The user can additionally provide information about the user (e.g., location, sex, age, height, social media interaction history, medical history, and/or the like), the user's schedule, and user preferences (e.g., location of a therapist, sex of the therapist, years of experience of the therapist, location of facility, time availability, and/or the like). The event-scheduling device can search in the first score table and the second score table to find a type of therapy that can best match the user's request by multiplying the scores in the first table with the scores in the second table and find the type of therapy that has the highest multiplication of the scores. In this example, the Trigger Point Massage has the highest value of 3.8 among multiplications of scores in the first score table and the second score table. The event-scheduling device can normalize the highest multiplication (e.g., 3.8) to an upper limit of scores (e.g., 5), and further add the normalized highest score to a set of therapist scores calculated separately for each therapist based on the third score table and the user preference (e.g., can add a factor for a therapist and/or location that matches user preferences such as, for example, location of a therapist, sex of the therapist, years of experience of the therapist, location of facility, time availability, and/or the like), to generate overall scores. The event-scheduling device can then generate the entries/recommendation (e.g., matching scores) to the user by listing therapists in order of a highest overall score to a lowest overall score.

In other implementations, any other suitable algorithm and/or method can be used to determine matching scores and/or recommendations. For example, as discussed above, one or more machine learning models can be used with various variables as input (e.g., scores from the first score table, the second score table and/or the third score table; user preferences; therapist and/or facility expertise, amenities, reviews and/or location; etc.).

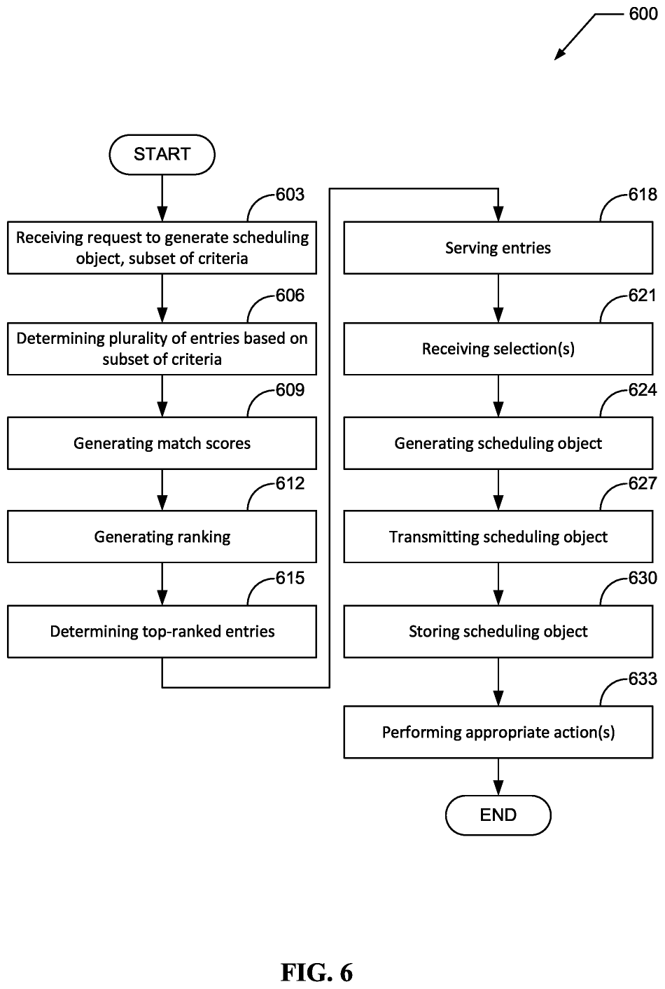

FIG. 6 shows an exemplary event scheduling process 600. In at least one embodiment, an event may include an entry that defines the event. For example, an entry for an event can include, but is not limited to, a particular facility, a particular practitioner, a particular treatment, a particular time, and/or a particular product. The event scheduling device 110 can perform the process 600 to process event criteria from a user, determine one or more entries that satisfy the event criteria, and generate one or more scheduling objects corresponding to the one or more entries.

At step 603, the process 600 includes receiving a request to generate a scheduling object and a subset of criteria. The request and the subset of criteria can be received by the event-scheduling device 110, for example, from a particular networking address. In another example, the event-scheduling device 110 receives the request and the subset of criteria from a user device 160 via a communication interface 112. In this example, the user device 160 can access the communication interface 112 via an installed application or a web or other network-based application. The

The event-scheduling device 110 can serve, at the particular networking address, a plurality of selectable fields defining a plurality of event scheduling criteria. The event-scheduling device 110 can receive selections to one or more of the plurality of selectable fields and, based thereon, determine a subset of the plurality of event scheduling criteria. The request to generate the scheduling object can include the subset of criteria. Alternatively, the event-scheduling device 110 can receive a first request to generate the scheduling object and a second request (or other communication) that includes the subset of the plurality of criteria.

The subset of criteria can include one or more of a time interval, a location, one or more treatment preferences, and one or more product preferences. Non-limiting examples of treatment preferences include body part, condition or malady, practitioner age, practitioner gender, treatment, treatment intensity, and treatment duration. Treatments and treatment intensities may include those described herein (see, for example, FIGS. 4-5) or any suitable treatment or treatment intensity. Non-limiting examples of product preferences include particular product types or brands, non-use of particular products (for example, palm oil-based products, products tested on animals, etc.), and product price levels. The location can include a current location of the user device 160, such as in the form of global positioning system (GPS) coordinates or another suitable location information format. In one example, the event-scheduling device 110 determines a location of the user device 160 based on the request. In another example, the user device 160 transmits a current location to the event-scheduling device 110.

The event-scheduling device 110 can receive the request by detecting an access event at a particular networking address. The event-scheduling device 110 can determine a location associated with the access event, such as, for example, a current location of a user device 160 that initiated the access event. The event-scheduling device 110 can determine, based on the location, one or more criteria and serve the one or more criteria at the particular networking address. For example, the event-scheduling device 110 determines facilities, treatments, practitioners, and products that are accessible within a predetermined proximity of the location (e.g., 50 miles, 100 miles, or any suitable value) and the event-scheduling device 110 generates and serves the plurality of criteria based on the determination.

At step 606, the process 600 includes determining a plurality of entries based on the subset of criteria. Determining the plurality of entries can include identifying combinations of a facility, practitioner, treatment, and/or time interval that satisfy one or more of the subset of criteria. The event-scheduling device 110 can query one or more therapist platforms 120, facility platforms 130, and/or a database 140 to determine, for example, available times, treatments, and/or products associated with a particular facility or practitioner. The event-scheduling device 110 can generate the entry based on data returned from the one or more queries. The event-scheduling device 110 can determine a particular treatment, for example, by mapping a specified body part and/or condition to a corresponding treatment. In one example, the event-scheduling device 110 retrieves, from the database 140, a mapping of historical treatments and body parts and/or conditions that may be improved thereby. Continuing the example, to identify a treatment for a particular body part and condition, the event-scheduling device 110 analyzes the mapping and determines at least one treatment that is known to improve the particular body part and/or address the condition.

At step 609, the process 600 includes generating at least one match score for each of the plurality of entries. The match score can refer to a likelihood that the entry satisfies the subset of criteria. The match score can refer to one or more historical ratings, or metrics derived therefrom, that are associated with an entry (e.g., or at least one element thereof, such as a particular facility, practitioner, or treatment). The event-scheduling device 110 can generate a match score for an entry by combining a plurality of historical ratings associated with the entry contents. Combining the plurality of historical ratings can refer to computing an average rating, a median rating, a mode of the plurality of historical ratings, or a weighted combination of the plurality of historical ratings. In one example, to generate a match score for a particular entry, the event-scheduling device 110 applies a weight value to each of the plurality of historical ratings associated therewith, thereby generating a plurality of weighted ratings. Continuing the example, the event-scheduling device 110 generates the match score by computing an average of the plurality of weighted ratings. The weight value applied to each historical rating can be predetermined or computed via one or more algorithms, machine learning models, or other suitable techniques.

The event-scheduling device 110 can perform steps 606-609 by searching in a plurality of ratings tables (see FIGS. 4-5) to find a type of therapy that can best match the subset of criteria selected by the user. The event-scheduling device 110 can generate match scores by multiplying the scores in the plurality of tables (e.g., a highest match score being associated with the type of therapy that has the highest multiplication of the scores).

The event-scheduling device 110 can generate, train, and execute one or more machine learning models to generate the match scores. In one example, the event-scheduling device 110 generates a training dataset including historical event-scheduling criteria and historical ratings associated with various entries that comply with at least a subset of the historical event scheduling criteria. Continuing the example, using the training dataset the event-scheduling device 110 trains one more machine learning models to generate entries that comply with the historical criteria and maximize the potential rating of the entry. In the same example, the event-scheduling device 110 uses the one or more trained machine learning models to estimate weight values that may be applied to the plurality of historical ratings associated with each entry of step 606. In another example, the event-scheduling device 110 trains a machine learning model to analyze historical entries and historical ratings associated therewith and, based on the analysis, estimate a weight value for each historical rating.

At step 612, the process 600 includes generating a ranking of the plurality of entries based on the respective match scores. For example, the event-scheduling device 110 creates a list of the plurality of entries ordered by descending match score. In another example, the event-scheduling device 110 generates a ranking by comparing the match score of each entry to one or more predetermined thresholds and assigning the entry to one of a plurality of segments based on the match score meeting (or failing to meet) the one or more predetermined thresholds. In another example, the event-scheduling device 110 determines a subset of the plurality of entries having a match score that satisfies a predetermined threshold. Continuing the example, the event-scheduling device 110 generates the ranking by ordering the entries of the subset according to descending likelihood score.

At step 615, the process 600 includes determining one or more top-ranked entries from the ranking. The event-scheduling device 110 can determine a particular number of top-ranked entries (e.g., 1, 2, 10 or any suitable number of entries). The request of step 603 can include a selection for a particular number of entries (for example, top three entries, top five entries, etc.). The event-scheduling device 110 can determine the top-ranked entries based on the selection.

At step 618, the process 600 includes serving the one or more top-ranked entries to the particular networking address and/or user device 160 from which the request was received at step 603. The event-scheduling device 110 can serve the one or more top ranked entries to the user device 160 via the communication interface 112. The event-scheduling device 110 can serve the one or more top-ranked entries at the particular networking address in the form of a list in which each entry in the list is a selectable field associated with a particular top-ranked entry. The event-scheduling device 110 can serve the match scores and/or historical ratings associated with the one or more top-ranked entries (e.g., thereby allowing a user to observe disaggregated metrics or ratings for each entry).

At step 621, the process 600 includes receiving a selection of one of the one or more top-ranked entries that were served at step 618. The event-scheduling device 110 can receive, via the particular networking address or the communication interface 112, a user's selection of a particular top-ranked entry.

At step 624, the process 600 includes generating a scheduling object based on the selected entry. Generating the scheduling object can include, for example, generating an appointment based on the selected entry and the subset of criteria. For example, the subset of criteria includes a particular time and date for which a user wishes to receive treatment. In this example, in response to receiving the user's selection of a particular entry, the particular entry including a particular facility, particular treatment, and particular practitioner. Continuing the example, the event-scheduling device 110 generates an appointment for the particular facility, particular practitioner, and particular treatment at the particular time and date. Generating the scheduling object can include generating a calendar scheduling file, such as, for example, “.ics,” “.ical,” “.ifb,” or “.icalendar.” In some embodiments, the event-scheduling device 110 determines a scheduling object file format based on the request (e.g., the request may include an election of a particular file format). In at least one embodiment, the event-scheduling device 110 determines an appropriate scheduling object file format based on the particular user device 160 from which the request and/or selections were received. For example, the event-scheduling device 110 determines that a user device 160 is an Apple Iphone™ and, in response, generates a scheduling object in a “.icalendar” file format.

At step 627, the process 600 includes transmitting the scheduling object to one or more of the particular networking address, the user device 160, a therapist platform 120 associated with the selected entry, or a facility platform 130 associated with the selected entry. The event-scheduling device 110 can transmit the scheduling object to the user device 160 via the communication interface 112. The event-scheduling device 110 can transmit the scheduling object to a user account associated with the user device 160. The event scheduling device 110 can generate and transmit an SMS text message, email, push notification, application alert, or other electronic communication that includes the scheduling object.

At step 630, the process 600 includes storing the scheduling object. The event-scheduling device 110 can store the scheduling object in one or more databases 140. The event-scheduling device 110 can cause the user device 160 to store the scheduling object in memory 161. The event-scheduling device 110 can cause the therapist platform 120 and/or the facility platform 130 to store the scheduling object in memory 121 or memory 131. The event-scheduling device 110 can configure a storage policy for the scheduling object. For example, the event-scheduling device 110 can configure a policy that causes the database 140 to store the scheduling object for a predetermined time period (e.g., 1 year, 3 years, 5 years, or any suitable interval). In another example, the event-scheduling device 110 configures a policy that causes the therapist platform 120 and/or facility platform 130 to generate and store a reminder associated with the selected entry (e.g., the reminder, when elapsed, causing the therapist platform 120 and/or facility platform 130 to generate an alert for the selected entry).

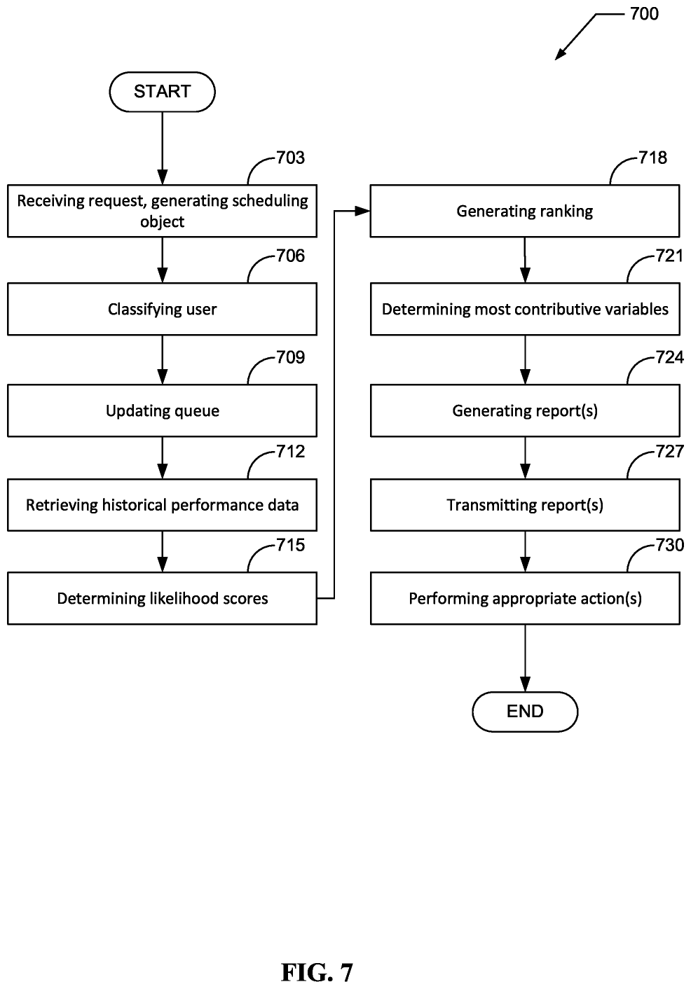

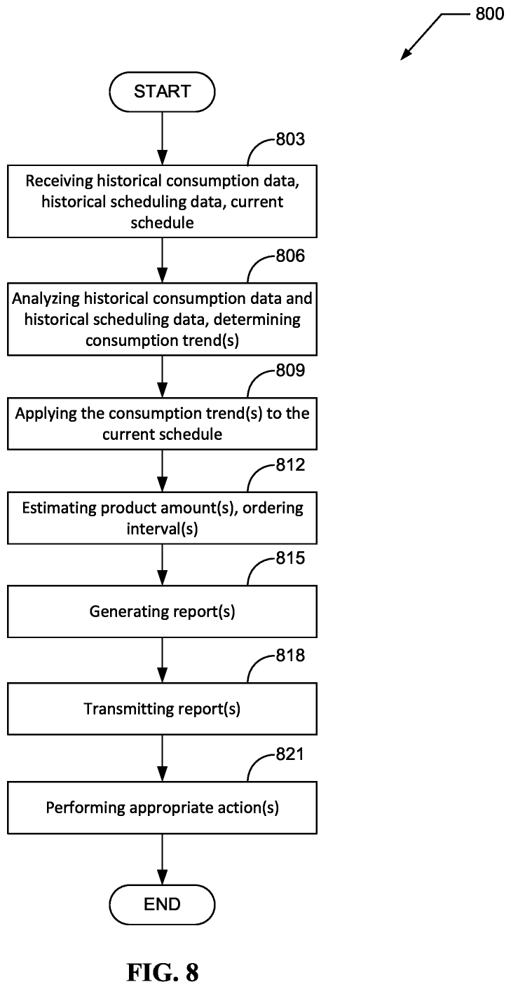

At step 633, the process 600 includes performing one or more appropriate actions. In one example, the event-scheduling device 110 performs a product estimation process 800 (see FIG. 8) to generate an estimated product amount and/or ordering interval for the facility or practitioner associated with the selected entry. In another example, the event-scheduling device 110 generates a counter based on an appointment time and date of the selected entry. In this example, the counter can refer to a timer that, when elapsed, causes the event-scheduling device 110 to generate and transmit an appointment alert to one or more of the user device 160, therapist platform 120, and facility platform 130. In another example, the event-scheduling device 110 performs a recommendation generation process 700 (see FIG. 7) to generate one or more recommendations for the facility and/or practitioner associated with the selected entry. In this example, the one or more recommendations can be directed towards improving customer experience and/or ratings for the corresponding facility or practitioner. In another example, the event-scheduling device 110 receives a command to cancel the scheduling object. Continuing this example, in response to the command, the event-scheduling device 110 generates and transmits an alert to the user device 160, therapist platform 120, and/or facility platform 130 associated with the scheduling object.

In another example, the event-scheduling device 110 receives a command to reschedule the scheduling object for a particular time interval. In this example, in response to the command, the event-scheduling device 110 performs another instance of the process 600 to determine a second scheduling object for replacement of the original scheduling object (e.g., the second scheduling object falling within the particular time interval). Continuing this example, the event-scheduling device 110 transmits an alert to one or more of the particular networking address, the user device 160, the therapist platform 120, and/or the facility platform 130, the alert including the second scheduling object and indicating the rescheduling of the original scheduling object.

Subsequent to the scheduling object expiring (e.g., referring to passage of an associated time and date), the event-scheduling device 110 can receive various data. For example, the event-scheduling device 110 receives, from the user device 160, therapist platform 120, and/or facility platform 130, a confirmation that an appointment associated with the scheduling object occurred (or did not occur). In another example, the event-scheduling device 110 receives feedback, such as, for example, ratings of the facility, practitioner, and/or treatment associated with the selected entry. In this example, the event-scheduling device 110 stores the feedback for use in future scheduling processes and/or transmits the feedback to a therapist platform 120 or facility platform 130 referenced thereby. In a similar example, in response to the feedback, the event-scheduling device 110 performs a recommendation process to generate suggestions for improving the facility, practitioner, and/or treatment (e.g., overall improvement or improvement for a particular user associated with the feedback).

In another example, the event-scheduling device 110 receives purchase data, the purchase data indicating one or more products or services purchased by a user or used by a facility or practitioner in association with the scheduling object. Continuing the example, the event-scheduling device stores the purchase list and/or performs an estimation process to generate an estimated product amount and/or ordering interval for a facility or practitioner associated with the purchase data.

FIG. 7 shows an exemplary recommendation generation process 700. The description of the process 700 may refer to generating recommendations for a plurality of entities; however, it can be understood and appreciated that the process 700 can be performed to generate recommendations for a single entity. According to one embodiment, the recommendation generated via the process 700 refers to a report (or other communication) that identifies one or more aspects of the entity that most heavily contributed to a likelihood score computed for the entity. In at least one embodiment, the likelihood score refers to a likelihood that an entity can satisfy a scheduling object (e.g., based on the entity meeting criteria defined by the scheduling object and/or based on historical ratings associated with the entity).

At step 703, the process 700 includes receiving a request and generating a scheduling object. In at least one embodiment, step 703 includes performing one or more event scheduling processes 600 (FIG. 6) to generate the scheduling object. The event-scheduling device 110 can receive the request from a user device 160. In some embodiments, the process 700 omits step 706 and, following step 703, the process 700 proceeds to step 709.

At step 706, the process 700 includes classifying a user associated with the request. The event-scheduling device 110 can determine a user associated with the request. The event-scheduling device 110 can classify the user into one of a plurality of segments based on the scheduling object. In some embodiments, at step 709, the event-scheduling device 110 retrieves the set of historical performance data based on the particular segment into which a user is classified. For example, the event-scheduling device 110 classifies the user into a segment associated with users that requested similar event scheduling criteria, purchased similar products or services, or demonstrated other similar factors, such as, for example, age, location, or gender. Classifying the user can include, for example, analyzing the scheduling object to generate a match score and assigning the user to one of a plurality of segments based on the match score, each of the plurality of segments being associated with a particular classification.

The event-scheduling device 110 can determine the user based on the request. For example, the event-scheduling device 110 determines a particular user identified in the request or who is associated with the user device 160 from which the request was received. In another example, the event-scheduling device 110 uniquely identifiers a user based on a device identifier corresponding to the user's user device 160. The device identifier can include, for example, a phone number, serial number, IP address, or MAC address.

The event-scheduling device 110 can generate the plurality of segments by analyzing historical data associated with a plurality of users. The event-scheduling device 110 can apply any suitable classification or segmentation technique, algorithm, or machine learning model to generate the plurality of segments. Non-limiting examples of machine learning techniques that may be applied include neural networks, linear regression, logistic regression, ordinary least squares regression, stepwise regression, multivariate adaptive regression splines, ridge regression, least-angle regression, locally estimated scatterplot smoothing, decision trees, random forest classification, support vector machines, Bayesian algorithms, hierarchical clustering, k-nearest neighbors, K-means, expectation maximization, association rule learning algorithms, learning vector quantization, self-organizing map, locally weighted learning, least absolute shrinkage and selection operator, elastic net, dimensionality reduction, and gradient boosting algorithms. As used herein, neural networks can generally include uni- or multilayer perceptron, convolutional neural network, recurrent neural network, long short-term memory networks, auto-encoders, deep Boltzman machine, deep belief network, back-propagation, stochastic gradient descent, Hopfield network, and radial basis function network.

The event-scheduling device 110 can generate a training dataset including historical data associated with a plurality of additional users. In some embodiments, the event-scheduling device 110 retrieves the historical data from a plurality of user devices 160, the therapist platform 120, the facility platforms 130, the database 140, or the social network server 150. The historical data can include, for example, historical scheduling objects, purchase histories, and/or feedback associated with the additional users. The event-scheduling device 110 can generate a machine learning model and, using the training dataset, train the machine learning model to generate the plurality of segments by analyzing the historical data and segmenting the plurality of additional users into the plurality of segments. The event-scheduling device 110 can execute the trained machine learning model on the scheduling object to classify the user into one of the plurality of segments. For example, the event-scheduling device 110 analyzes historical scheduling objects and purchase histories for each of the plurality of additional users and generates groupings of users based on similarities in the historical scheduling objects and purchase histories associated therewith. In this example, the event-scheduling device 110 generates a first segment of “light spend” users, a second segment of “normal spend” users, and a third segment of “high spend” users. Continuing the example, the event-scheduling device executes the trained machine learning model on the scheduling object and outputs an assignment classifying the scheduling object and, thereby, the user into the third segment of “high spend” users.

At step 709, the process 700 includes updating a queue based on the scheduling object. The queue can be associated with a particular service, such as, for example, a particular treatment (or class of treatments), a particular body cart or condition, a particular product used in a treatment. The queue can be a time series record of scheduling objects associated with the particular service. Updating the queue based on the scheduling object can include, for example, determining particular data from the scheduling object and generating an entry to the queue, the entry including the particular data. The particular data determined from the scheduling object can include, but is not limited to, a time interval (e.g., a time and date at which an appointment may take place), a location preference, facility preferences, practitioner preferences, treatment preferences, and product preferences. In one example, the event-scheduling device 110 determines, from a scheduling object, an appointment date of December 8^{th} at 12 PM EST, a location preference for 50 miles or less from Sandy Springs, Ga., USA, a male practitioner preference, and a treatment preference for Reiki. Continuing the example, the event-scheduling device 110 determines the scheduling object is associated with a Reiki service and, in response, updates a Reiki queue to include a new entry (e.g., the new entry including the data that was determined from the scheduling object).

In at least one embodiment, the event-scheduling device 110 retrieves the queue from memory 111 or from one or more databases 140. The event-scheduling device 110 can store the updated queue in memory 111 or at the database 140.

At step 712, the process 700 includes retrieving a set of historical performance data for a plurality of entities. The historical performance data can include, for each entity, facilities, practitioners, treatments, and/or products associated with the entity, historical scheduling objects associated with the entity, historical ratings associated with the entity, and a current schedule associated with the entity. The event-scheduling device 110 can generate the set of historical performance data by collecting the historical performance data (or a subset thereof) from one or more therapist platforms 120, facility platforms 130, and/or databases 140. In some embodiments, the event-scheduling device 110 can retrieve the set of historical performance data from memory 111 or from the database 140. The event-scheduling device 110 can retrieve the set of historical performance data based on a particular segment into which the user and/or scheduling object is classified. In one example, the event-scheduling device 110 classifies the user into a “low spend” segment and, based thereon, retrieves historical performance data associated with additional users that were classified into the “low spend” segment. In another example, the event-scheduling device 110 classifies the user into a “high end product” segment and, based thereon, retrieves historical performance data associated with the purchase of products classified as “high end” (e.g., based on their price, scarcity, popularity, etc.).

At step 715, the process 700 includes determining a likelihood score for each of the plurality of entities, respectively. The likelihood score can refer to a likelihood of the particular entity satisfying the scheduling object.

The event-scheduling device 110 can generate a likelihood score for an entry by generating a plurality of variables based on the historical performance data and the scheduling object and determining the likelihood score based on a weighted combination of the plurality of variables. The event-scheduling device 110 can generate a variable, for example, by assigning a historical performance data element to one of a plurality of segments and associating the variable with a particular value based on the assigned segment. It can be understood and appreciated that the event-scheduling device 110 can assign values to each variable by comparing the variable contents to a set of corresponding segments, each segment being associated with a particular scaled value. According to one embodiment, by mapping historical performance data elements to scaled variable values, the event-scheduling device 110 may enable quantitative analysis of multiple data points of differing format and type.

In one example, the event-scheduling device 110 generates a proximity variable by computing a distance between an entity's facility and a location defined by the scheduling object. Continuing the example, the event-scheduling device 110 compares the distance to a first distance range associated with a “distant” segment, a second distance range associated with a “drivable” segment, and a third distance range associated with a “nearby” segment. In the same example, the event-scheduling device 110 determines that the distance is within the third distance range and, in response, assigns the proximity variable to the “nearby” segment. Continuing the example, the event-scheduling device 110 assigns the proximity variable a value of 3.0 based on the assignment to the “nearby” segment. In an alternate example, the event-scheduling device 110 determines that the distance within the second distance range, assigns the proximity variable to the “drivable” segment, and assigns the proximity variable a value of 2.0. In another alternate example, the event-scheduling device 110 determines that the distance within the first distance range, assigns the proximity variable to the “distant” segment, and assigns the proximity variable a value of 1.0. It can be understood and appreciated that the event-scheduling device 110 can use any suitable set of scaled values for generating variables described herein and that the values of 1.0-3.0 are provided by way of example and for the purposes of describing an exemplary embodiment of the process 700.

The event-scheduling device 110 can assign an initial weight value to each variable. The event-scheduling device 110 can generate an initial likelihood score by combining the variables according to their initial weight value. The event-scheduling device 110 can compute an error metric associated with the initial likelihood score and adjust one or more of the initial weight values toward reducing the error metric. The error metric can refer to a root mean squared error computed by comparing the likelihood score to one or more historical likelihood scores associated with criteria similar (or identical) to criteria with which the scheduling object is associated. Non-limiting examples of error metric include classification accuracy, logarithmic loss, confusion matrix, area under curve, F1 score, mean absolute error, and mean squared error. The event-scheduling device 110 can iteratively perform weight adjustment until the error metric meets a predetermined threshold. The event-scheduling device 110 can configure the weight values associated with the threshold-satisfying error metric as the final version of weight values that are used to compute the likelihood score.

The event-scheduling device 110 can generate, train, and execute one or more machine learning models to generate the variables and/or generate the likelihood score for each variable. For example, the event-scheduling device 110 can initialize and optimize a weight value for each variable.

At step 718, the process 700 includes generating a ranking of the plurality of entities based on the likelihood scores. For example, the event-scheduling device 110 creates a list of the plurality of entities ordered by descending likelihood score. In another example, the event-scheduling device 110 generates a ranking by comparing the likelihood score of each entity to one or more predetermined thresholds and assigning the entity to one of a plurality of segments based on the likelihood score meeting (or failing to meet) the one or more predetermined thresholds. In another example, the event-scheduling device 110 determines a subset of the plurality of entities having a likelihood score that satisfies a predetermined threshold. Continuing the example, the event-scheduling device 110 generates the ranking by ordering the entities of the subset by descending likelihood score.

At step 721, the process 700 includes determining, for each of the plurality of entities, one or more aspects of the historical data that most contributed to the corresponding likelihood score. For example, the event-scheduling device 110 determines, for each likelihood score, at least one variable that most positively contributed to the likelihood score and at least one variable that most negatively contributed to the likelihood score. The event-scheduling device 110 can analyze the weight value assigned to each variable during computation of the likelihood score. The event-scheduling device 110 can determine a most positively contributing variable by identifying a variable associated with the highest weight value (or, in particular, the highest positive weight value). The event-scheduling device 110 can determine a most negatively contributing variable by identifying a variable associated with the lowest weight value (or, if applicable, the greatest negative weight value).

In one example, the event-scheduling device 110 determines that, for a particular entity, a facility rating variable was assigned the greatest positive weight value and a practitioner rating variable was assigned the greatest negative weight value. In this example, the weight value of the facility rating variable may indicate that the quality of the entity's facility may be the most important current factor for attracting and maintaining business that corresponds to the scheduling object. In the same example, the weight value of the practitioner rating variable may indicate that the quality of the practitioner(s) at the facility may be the greatest current impediment to attracting and maintaining business that corresponds to the scheduling object.

At step 724, the process 700 includes generating a report (or other suitable communication) for each of the plurality of entities, respectively. The report can include the one or more aspects determined at step 721. For example, the report includes at least one variable that most positively contributed to the entity's likelihood score and at least one variable that most negatively contributed to the entity's likelihood score. The event-scheduling device 110 can generate the report as a user interface that can be rendered on a display. The event-scheduling device 110 can generate the report as an electronic communication, such as, for example, an email, text message, or push notification. The event-scheduling device 110 can generate the report in any suitable file format, such as, for example, .PDF, .txt, or .docx. The event-scheduling device 110 can generate a spreadsheet (e.g., a .xls, .xlsx, .csv, or other spreadsheet file) that includes the likelihood score, the one or more aspects that most heavily contributed to the likelihood score, and/or the historical data and scheduling object from which the likelihood score was determined.

At step 727, the process 700 includes transmitting the reports to the respective entity. The event-scheduling device 110 can transmit the report, for example, to a therapist platform 120 and/or a facility platform 130 associated with the entity. In some embodiments, the entity itself refers to a particular therapist platform 120 or the facility platform 130.

At step 730, the process 700 includes performing one or more appropriate actions. In one example, the event-scheduling device 110 stores the reports in memory 111 and/or at one or more databases 140. In another example, the event-scheduling device 110 serves each report to a respective web portal (e.g., or other online environment) at which the corresponding entity may view the report. In another example, the event-scheduling device 110 generates a recommendation for one or more actions that an entity may perform to potentially improve the entity's likelihood score. For example, the event-scheduling device 110 determines that an entity's facility location most negatively contributed to the entity's likelihood score due to the facility location being beyond a predetermined proximity from a location associated with the scheduling object. Continuing the example, the event-scheduling device 110 generates a recommendation to improve the entity's likelihood score by opening a new facility at a second location that is closer to the location associated with the scheduling object.

The event-scheduling device 110 can update a waitlist based on the scheduling object of step 703. For example, the event-scheduling device 110 retrieves a listing of additional scheduling objects that are not associated with a particular entity and updates the listing the listing to include the scheduling object. The event-scheduling device 110 can determine at least one of the plurality of entries for which an associated likelihood fails to satisfy a predetermined threshold associated with the scheduling object. The event-scheduling device 110 can determine at least one entity that was nearest to, but excluded from, a plurality of top-ranked entities of the ranking. The event-scheduling device 110 can determine at least one low-ranked entity from the ranking. For the at least one entity, the event-scheduling device 110 can determine a likelihood score for more of the additional scheduling objects from the waitlist. The event-scheduling device 110 can determine at least one of the additional scheduling objects for which the at least one entity demonstrates a likelihood score that satisfies a second predetermined threshold associated with the at least one of the additional scheduling objects. The event-scheduling device 110 can transmit the at least one of the additional scheduling objects to the at least one entity. The event-scheduling device 110 can receive, from the entity, a confirmation of the at least one of the additional scheduling objects. In response to receiving the confirmation, the event-scheduling device 110 can remove the at least one of the additional scheduling objects from the waitlist.

FIG. 8 shows an exemplary product estimation process 800. In various embodiments, the event-scheduling device 110 performs the process 800 to estimate an amount of product that an entity may need to have on hand to satisfy a scheduling object. For example, a current schedule of an entity includes a plurality of scheduling objects that include use of a particular hair product. In this example, the product service 108 estimates an amount of the particular hair product that may be required to satisfy each of the plurality of scheduling objects. Continuing the example, the product service 108 determines an ordering interval during with the estimated amount of the particular hair product may be ordered to ensure its availability for each of the plurality of scheduling objects.

At step 803, the process 800 includes receiving historical consumption data, historical scheduling data, and a current schedule associated with one or more entities. The entity can refer to a particular facility, a particular practitioner, or a combination thereof. The current schedule can include one or more scheduling objects associated with the entity. The historical scheduling data can include historical scheduling objects and ratings associated with the entity. The historical consumption data can include, for example, purchase lists for a facility or practitioner (e.g., the purchase list including historical prices and quantities for various products) or inventory data for tracking consumption of various products over time.

The event-scheduling device 110 can receive the historical consumption data, historical scheduling data, and current schedule from a therapist platform 120 or a facility platform 130. The product service 108 can transmit a request for the historical consumption data, historical scheduling data, and/or current schedule to the therapist platform 120 or facility platform 130. In one example, the product service 108 determines that a counter meets a predetermined threshold and, in response, transmits the request. The counter can refer to a time counter (for example, a number of days since a product purchase date) or an instance counter (for example, a number of scheduling objects generated and/or completed by the corresponding facility or practitioner). The product service 108 can store historical consumption data, historical scheduling data, and/or current schedule in memory 111 or at the database 140.

At step 806, the process 800 includes analyzing the historical consumption data, historical scheduling data, and current schedule and determining one or more consumption trends based on the analysis. The consumption trend can refer to a formula or equation that, given an input of a current schedule, estimates an amount of product that may be necessary to satisfy the current schedule. The product service 108 can determine, based on the historical consumption data and the historical scheduling data, a historical rate at which a particular product was consumed over the course of satisfying a plurality of historical scheduling objects. The product service 108 can apply the historical rate to the current schedule to estimate the consumption trend.

The product service 108 can determine an average amount of product that was used to satisfy a particular number of historical scheduling objects (e.g., the particular number being similar to a number of scheduling objects associated with the current schedule). The product service 108 can generate the consumption trend based on relating the average amount of product to an input number of scheduling objects.

The product service 108 can adjust the consumption trend based on a segment into which the current schedule (e.g., or scheduling objects and/or users associated therewith) were classified. In one example, the event-scheduling device assigns a current schedule to a “heavy product use” segment and, in response, the product service 108 adjusts a consumption trend such that the consumption trend estimates a comparatively greater amount of the product required to satisfy the current schedule. The product service 108 can determine a consumption trend for each of a plurality of segments, respectively.

At step 809, the process 800 includes applying the one or more consumption trends to the current schedule. The product service 108 can apply the consumption trend to the current schedule, for example, by determining one or more input variables from the current schedule, such as, for example, a total number of schedule objects and one or more ratios of schedule objects (or associated users) that were assigned to a particular segment (e.g., a heavy product use segment, light product use segment, etc.). The product service 108 can assign the current schedule to a particular segment, determine one of a plurality of consumption trends that corresponds to the particular segment, and apply the determined consumption trend to the current schedule.

At step 812, the process 800 includes estimating a product amount and an ordering interval for the entity based on applying the one or more consumption trends to the current schedule. The product service 108 can generate, as an output of applying the consumption trend, an estimated product amount. The product service 108 can apply a predetermined safety factor to the estimated product amount (e.g., to further ensure sufficient product is available for the current schedule). For example, the product service 108 automatically increases an estimated product amount by 3%, 5%, 10%, or another suitable value.

The product service 108 can estimate an ordering interval based on the estimated product amount. The product service 108 can analyze a delivery history for the product and, based thereon, estimate an average delivery time for the product. The product service 108 can compare the average delivery time to a date of the earliest scheduling object of the current schedule. Based on the comparison, the product service 108 can determine a particular date by which the estimated product amount should be ordered to ensure delivery by the date of the earliest scheduling object. The product service 108 can generate the ordering interval as a period extending from a current date to the particular date. The product service 108 can apply a predetermined safety factor to the ordering interval (e.g., to further ensure sufficient product is available for the current schedule). For example, the product service 108 automatically subtracts a 1, 2, 3 or any suitable number of days from the particular date to reduce the ordering interval. In some embodiments, the product service 108 subtracts a configurable number of days. The product service 108 can estimate the product amount safety factor and/or the ordering interval safety factor based on an analysis of the historical consumption data and historical scheduling data. For example, the product service 108 applies the consumption trend to the historical consumption data and historical scheduling data and determines a frequency of instances and/or a degree to which the consumption trend failed to accurately estimate the required product amount (e.g., or ordering interval). Continuing the example, the product service 108 determines the safety factor by identifying an adjustment or offset that may be applied to the consumption trend (e.g., or the output thereof) to increase the output accuracy to a predetermined accuracy threshold.

At step 815, the process 800 includes generating a report including the product amount and the ordering interval. The event-scheduling device 110 can generate the report as a user interface that can be rendered on a display. The event-scheduling device 110 can generate the report as an electronic communication, such as, for example, an email, text message, or push notification. The event-scheduling device 110 can generate the report in any suitable file format, such as, for example, .PDF, .txt, or .docx. The event-scheduling device 110 can generate a spreadsheet (e.g., a .xls, .xlsx, .csv, or other spreadsheet file) that includes the estimated product amount, the ordering interval, an estimated cost of the product amount, and/or a provider of the product (for example, a particular commercial supplier). In some embodiments, a cost of the product amount is referred to as a product amount value.

At step 818, the process 800 includes transmitting the report to the entity. The event-scheduling device 110 can transmit the report, for example, to a therapist platform 120 and/or a facility platform 130 associated with the entity. The product service 108 can determine that a current interval is within a predetermined range of the ordering interval and, in response, transmit the report to the entity. The predetermined range can be, for example, 1 day, 2 weeks, 1 month, or any suitable and/or configured value. For example, the product service 108 determines an ordering interval of December 8^{th}. Continuing the example, the product service 108 determines that a current date, December 2^{nd}, is within one week of the ordering interval and, in response, transmits the report to the entity. The product service 108 can determine that a current product amount associated with the entity fails to meet a predetermined threshold. In response to determining the current product amount fails to meet the predetermined threshold, the product service 108 automatically transmits the report to the entity.

At step 821, the process 800 includes performing one or more appropriate actions. The product service 108 can store the estimated product amount and/or the ordering interval in memory 111 and/or at the database 140. The product service 108 can generate a counter based on the ordering interval. The counter can be configured to count a number of days preceding the ordering interval. In response to the counter being within a predetermined range of the ordering interval, the product service 108 can generate and transmit a product order reminder to the entity. The product service 108 can generate a scheduling object based on the ordering interval and transmit the scheduling object to the entity.

The product service 108 can determine one or more suppliers for the supplying product at the estimated product amount. The product service 108 can estimate a cost of purchasing the estimated product amount from the supplier. For example, the product service 108 transmits a query to a supplier system, the query including the estimated product amount and an identifier corresponding to the product (e.g., a product name, brand, or other identifying information). Continuing the example, in response to the query, the product service 108 receives a cost estimate from the supplier system. The product service 108 can include in the report the estimated cost of purchasing the product. In some embodiments, the product service 108 automatically submits an order to the supplier for the estimated product amount. In at least one embodiment, the order includes the estimated product amount and information associated with the entity, such as, for example, a name, address, email, or account number.

According to a first aspect, a method, comprising: A) receiving, via at least one computing device, historical consumption data, historical scheduling data, and a current docket associated with at least one entity; B) analyzing, via the at least one computing device, the historical consumption data and the historical scheduling data to determine at least one consumption rate; C) applying, via the at least one computing device, the at least one consumption rate to the at least one current docket; D) based on applying the at least one consumption rate, calculating, via the at least one computing device, at least one resource amount and at least one ordering interval; E) generating, via the at least one computing device, a communication comprising the at least one resource amount and the at least one ordering interval; and F) transmitting, via the at least one computing device, the communication to the at least one entity.

According to a further aspect, the method of the first aspect or any other aspect, further comprising determining that a current interval is within a predetermined range of the at least one ordering interval, wherein the communication is transmitted to the at least one entity in response to the determination.

According to a further aspect, the method of the first aspect or any other aspect, further comprising determining one or more providers for the at least one resource amount.

According to a further aspect, the method of the first aspect or any other aspect, further comprising calculating a value of the at least one resource amount.

According to a further aspect, the method of the first aspect or any other aspect, further comprising determining that, based on the at least one consumption rate, a projected resource amount is less than a predetermined threshold, wherein the communication is transmitted to the at least one entity in response to the determination.

According to a second aspect, a system, comprising: A) at least one database; and B) at least one computing device configured to: 1) receive historical consumption data, historical scheduling data, and a current docket associated with at least one entity; 2) analyze, via the at least one computing device, the historical consumption data and the historical scheduling data to determine at least one consumption rate; 3) generate, via the at least one computing device, a comparison of the at least one consumption rate to the at least one current docket; 4) based on the comparison, calculate, via the at least one computing device, at least one resource amount and at least one ordering interval; 5) generate a communication comprising the at least one resource amount and the at least one ordering interval; and 6) record the communication at the at least one database.

According to a further aspect, the system of the second aspect or any other aspect, wherein the at least one computing device is configured to transmit the communication to the at least one entity.

According to a further aspect, the system of the second aspect or any other aspect, wherein the at least one computing device is configured to determine that a current interval is within a predetermined range of the at least one ordering interval, wherein the communication is transmitted to the at least one entity in response to the determination.

According to a further aspect, the system of the second aspect or any other aspect, wherein the at least one computing device is configured to determine that, based on the at least one consumption rate, a projected resource amount is less than a predetermined threshold, wherein the communication is transmitted to the at least one entity in response to the determination.

According to a third, a non-transitory computer-readable storage medium storing a plurality of instructions executable by at least one processor, the plurality of instructions when executed by the at least one processor cause the at least one processor to: A) receive, via at least one computing device, historical consumption data, historical scheduling data, and a current docket associated with at least one entity; B) analyze, via the at least one computing device, the historical consumption data and the historical scheduling data to determine at least one consumption rate; C) calculate a resource usage amount by applying the consumption rate to the current docket; D) generate at least one ordering interval based on the resource usage amount and the current docket; E) generate a communication comprising the resource usage amount and the at least one ordering interval; and F) transmit the communication to the at least one entity.

According to a further aspect, the non-transitory computer-readable storage medium of the third aspect or any other aspect, wherein the plurality of instructions, when executed by the at least one processor, further cause the at least one processor to determine that a current interval is within a predetermined range of the at least one ordering interval, wherein the communication is transmitted to the at least one entity in response to the determination.

According to a further aspect, the non-transitory computer-readable storage medium of the third aspect or any other aspect, wherein the plurality of instructions, when executed by the at least one processor, further cause the at least one processor to: A) generate a scheduling object based on the at least one ordering interval; and B) transmit the scheduling object to the at least one entity.

According to a further aspect, the non-transitory computer-readable storage medium of the third aspect or any other aspect, wherein the plurality of instructions, when executed by the at least one processor, further cause the at least one processor to determine that, based on the at least one consumption rate, a projected resource amount is less than a predetermined threshold, wherein the communication is transmitted to the at least one entity in response to the determination.

According to a fourth aspect, a method, comprising: A) generating, via at least one computing device, a scheduling object in response to a request; B) updating, via the at least one computing device, a queue based on the scheduling object, the queue corresponding to at least one service; C) retrieving, via the at least one computing device, a set of historical performance data for a plurality of entities associated with the at least one service; D) determining, via the at least one computing device, a likelihood score for each of the plurality of entities based on the set of historical performance data and the scheduling object, wherein the likelihood score refers to a likelihood of satisfying the scheduling object; E) ranking, via the at least one computing device, the plurality of entities based on the likelihood score; F) determining, via the at least one computing device and for each of the plurality of entities: 1) at least one first respective aspect of the historical performance data that most negatively contributed to the likelihood score; and 2) at least one second respective aspect of the historical performance data that most positively contributed to the likelihood score; and G) generating, via the at least one computing device, a plurality of communications individually corresponding to a respective one of the plurality of entities, each of the plurality of communications comprising an instruction based on the at last one first respective aspect and the at least one second respective aspect.

According to a further aspect, the method the fourth aspect or any other aspect, further comprising: A) determining a user associated with the request; and B) classifying the user into one of a plurality of segments based on the scheduling object, wherein the set of historical performance data for the plurality of entities is obtained based on the one of the plurality of segments into which the user is classified.

According to a further aspect, the method of the fourth aspect or any other aspect, wherein classifying the user into the one of the plurality of segments comprises: A) generating a training dataset comprising historical data associated with a plurality of users; B) training a machine learning model to generate the plurality of segments by analyzing the historical data and segmenting subsets of the plurality of users into the plurality of segments; and C) executing the trained machine learning model on the scheduling object to classify the user into the one of the plurality of segments.

According to a further aspect, the method of the fourth aspect or any other aspect, wherein the machine learning model is a random forest classification model.

According to a further aspect, the method of the fourth aspect or any other aspect, wherein the machine learning model is a neural network.

According to a further aspect, the method of the fourth aspect or any other aspect, further comprising transmitting, via the at least one computing device, each of the plurality of communications to the corresponding one of the plurality of entities.

According to a further aspect, the method of the fourth aspect or any other aspect, further comprising: A) updating a hold list based on the scheduling object; B) determining at least one entity of the plurality of entities for which the likelihood score cannot satisfy a predetermined threshold associated with the scheduling object; and C) determining, for the at least one entity, at least one second scheduling object from the hold list, wherein the at least one entity demonstrates a second likelihood score that satisfies a second predetermined threshold associated with the at least one second scheduling object.

According to a fifth aspect, a system, comprising: A) at least one database; and B) at least one computing device operatively connected to the at least one database, wherein the at least one computing device is configured to: 1) generate a scheduling object in response to a request; 2) update a queue based on the scheduling object, the queue corresponding to at least one service; 3) obtain a set of historical performance data for a plurality of entities associated with the at least one service; 4) determine a likelihood score for each of the plurality of entities based on the set of historical performance data and the scheduling object, wherein the likelihood score refers to a likelihood of satisfying the scheduling object; 5) rank the plurality of entities based on the likelihood score; 6) determine, for each of the plurality of entities: i) at least one first respective aspect of the historical performance data that most negatively contributed to the likelihood score; and ii) at least one second respective aspect of the historical performance data that most positively contributed to the likelihood score; and 7) generate a plurality of communications individually corresponding to a respective one of the plurality of entities, each of the plurality of communications comprising an instruction based on the at last one first respective aspect and the at least one second respective aspect.

According to a further aspect, the system of the fifth aspect or any other aspect, wherein the at least one computing device is configured to: A) determine a user associated with the request; and B) classify the user into one of a plurality of segments based on the scheduling object, wherein the set of historical performance data for the plurality of entities is obtained based on the one of the plurality of segments into which the user is classified.

According to a further aspect, the system of the fifth aspect or any other aspect, wherein the at least one computing device is configured to classify the user into the one of the plurality of segments by: A) generating a training dataset comprising historical data associated with a plurality of users; B) training a machine learning model to generate the plurality of segments by analyzing the historical data and segmenting subsets of the plurality of users into the plurality of segments; and C) executing the trained machine learning model on the scheduling object to classify the user into the one of the plurality of segments.

According to a further aspect, the system of the fifth aspect or any other aspect, wherein the at least one computing device is configure to transmit each of the plurality of communications to the corresponding one of the plurality of entities.

According to a further aspect, the system of the fifth aspect or any other aspect, wherein the at least one computing device is configured to: A) update a hold list based on the scheduling object; B) determine at least one entity of the plurality of entities for which the likelihood score cannot satisfy a predetermined threshold associated with the scheduling object; and C) determine, for the at least one entity, at least one second scheduling object from the hold list, wherein the at least one entity demonstrates a second likelihood score that satisfies a second predetermined threshold associated with the at least one second scheduling object.

According to a sixth aspect, a non-transitory computer-readable storage medium storing a plurality of instructions executable by at least one processor, wherein the plurality of instructions, when executed by the at least one processor, cause the at least one processor to: A) generate a scheduling object in response to a request; B) update a queue based on the scheduling object, the queue corresponding to at least one service; C) obtain a set of historical performance data for a plurality of entities associated with the at least one service; D) determine a likelihood score for each of the plurality of entities based on the set of historical performance data and the scheduling object, wherein the likelihood score refers to a likelihood of satisfying the scheduling object; E) rank the plurality of entities based on the likelihood score; F) determine, for each of the plurality of entities: 1) at least one first respective aspect of the historical performance data that most negatively contributed to the likelihood score; and 2) at least one second respective aspect of the historical performance data that most positively contributed to the likelihood score; and G) generate a plurality of communications individually corresponding to a respective one of the plurality of entities, each of the plurality of communications comprising an instruction based on the at last one first respective aspect and the at least one second respective aspect.

According to a further aspect, the non-transitory computer-readable storage medium of the sixth aspect or any other aspect, wherein the plurality of instructions, when executed by the at least one processor, further cause the at least one processor to: A) determine a user associated with the request; and B) classify the user into one of a plurality of segments based on the scheduling object, wherein the set of historical performance data for the plurality of entities is obtained based on the one of the plurality of segments into which the user is classified.

According to a further aspect, the non-transitory computer-readable storage medium of the sixth aspect or any other aspect, wherein the plurality of instructions, when executed by the at least one processor, further cause the at least one processor to classify the user into the one of the plurality of segments by: A) generating a training dataset comprising historical data associated with a plurality of users; B) training a machine learning model to generate the plurality of segments by analyzing the historical data and segmenting subsets of the plurality of users into the plurality of segments; and C) executing the trained machine learning model on the scheduling object to classify the user into the one of the plurality of segments.

According to a further aspect, the non-transitory computer-readable storage medium of the sixth aspect or any other aspect, wherein the plurality of instructions, when executed by the at least one processor, further cause the at least one processor to transmit each of the plurality of communications to the corresponding one of the plurality of entities.

According to a further aspect, the non-transitory computer-readable storage medium of the sixth aspect or any other aspect, wherein the plurality of instructions, when executed by the at least one processor, further cause the at least one processor to: A) update a hold list based on the scheduling object; B) determine at least one entity of the plurality of entities for which the likelihood score cannot satisfy a predetermined threshold associated with the scheduling object; and C) determine, for the at least one entity, at least one second scheduling object from the hold list, wherein the at least one entity demonstrates a second likelihood score that satisfies a second predetermined threshold associated with the at least one second scheduling object.

According to a further aspect, the non-transitory computer-readable storage medium of the sixth aspect or any other aspect, wherein the plurality of instructions, when executed by the at least one processor, further cause the at least one processor to determine the likelihood score for each of the plurality of entities based on one or more additional scheduling objects from the queue.

Embodiments of event scheduling systems (e.g., event scheduling system 100) described herein can receive, analyze, and/or transmit data related to information about a user, a set of indications of expertise of a set of therapists, a set of indications associated with types of therapies, a set of indications of amenities and/or capabilities in a set of facilities, a first time schedule associated with the user, a second time schedule associated with the set of therapists, a third time schedule associated with the set of facilities and/or any other suitable information. In some embodiments, the event scheduling systems can additionally receive, analyze, and transmit data related to a location of a user (e.g., by using Global Positioning System (GPS) coordinates of the user device, obtaining an Internet Protocol (IP) address of a user device, requesting information from a user of the user device, accessing an address on an account associated with a user or user device, and/or the like), a location of a therapist (e.g., by parsing information about the practice registration of a computing device of the therapist, using GPS coordinates of the computing device of the therapist, obtaining an IP address of the computing device of the therapist, requesting information from a user of the computing device of the therapist, accessing an address on an account associated with a therapist, and/or the like), a location of a facility (e.g., by using GPS coordinates of a computing device of the facility, obtaining an IP address of the computing device of the facility, requesting information from a user of the computing device of the facility, accessing an address on an account associated with the facility, and/or the like).

Some embodiments described herein relate to methods. It should be understood that such methods can be computer-implemented methods (e.g., instructions stored in memory and executed on processors). Where methods described above indicate certain events occurring in a certain order, the ordering of certain events can be modified. Additionally, certain of the events can be performed repeatedly, concurrently in a parallel process when possible, as well as performed sequentially as described above. Furthermore, certain embodiments can omit one or more described events.

Examples of computer code include, but are not limited to, micro-code or micro-instructions, machine instructions, such as produced by a compiler, code used to produce a web service, and files containing higher-level instructions that are executed by a computer using an interpreter. For example, embodiments can be implemented using Python, R, Java, C++, or other programming languages (e.g., object-oriented programming languages) and development tools. Additional examples of computer code include, but are not limited to, control signals, encrypted code, and compressed code.

The drawings primarily are for illustrative purposes and are not intended to limit the scope of the subject matter described herein. The drawings are not necessarily to scale; in some instances, various aspects of the subject matter disclosed herein can be shown exaggerated or enlarged in the drawings to facilitate an understanding of different features. In the drawings, like reference characters generally refer to like features (e.g., functionally similar and/or structurally similar elements).

To address various issues and advance the art, the entirety of this application (including the Cover Page, Title, Headings, Background, Summary, Brief Description of the Drawings, Detailed Description, Embodiments, Abstract, Figures, Appendices, and otherwise) shows, by way of illustration, various embodiments in which the embodiments can be practiced. The advantages and features of the application are of a representative sample of embodiments only, and are not exhaustive and/or exclusive. They are presented to assist in understanding and teach the embodiments.

Also, no inference should be drawn regarding those embodiments discussed herein relative to those not discussed herein other than it is as such for purposes of reducing space and repetition. For instance, it is to be understood that the logical and/or topological structure of any combination of any program components (a component collection), other components and/or any present feature sets as described in the figures and/or throughout are not limited to a fixed operating order and/or arrangement, but rather, any disclosed order is an example and equivalents, regardless of order, are contemplated by the disclosure.

Various concepts can be embodied as one or more methods, of which at least one example has been provided. The acts performed as part of the method can be ordered in any suitable way. Accordingly, embodiments can be constructed in which acts are performed in an order different than illustrated, which can include performing some acts simultaneously, even though shown as sequential acts in illustrative embodiments. Put differently, it is to be understood that such features can not necessarily be limited to a particular order of execution, but rather, any number of threads, processes, services, servers, and/or the like that can execute serially, asynchronously, concurrently, in parallel, simultaneously, synchronously, and/or the like in a manner consistent with the disclosure. As such, some of these features can be mutually contradictory, in that they cannot be simultaneously present in a single embodiment. Similarly, some features are applicable to one aspect of the innovations, and inapplicable to others.

It should be understood that advantages, embodiments, examples, functional, features, logical, operational, organizational, structural, topological, and/or other aspects of the disclosure are not to be considered limitations on the disclosure as defined by the embodiments or limitations on equivalents to the embodiments. Depending on the particular desires and/or characteristics of an individual and/or enterprise user, database configuration and/or relational model, data type, data transmission and/or network framework, syntax structure, and/or the like, various embodiments of the technology disclosed herein can be implemented in a manner that enables a great deal of flexibility and customization as described herein.

All definitions, as defined and used herein, should be understood to control over dictionary definitions, definitions in documents incorporated by reference, and/or ordinary meanings of the defined terms.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements.

This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

From the foregoing, it will be understood that various aspects of the processes described herein are software processes that execute on computer systems that form parts of the system. Accordingly, it will be understood that various embodiments of the system described herein are generally implemented as specially configured computers including various computer hardware components and, in many cases, significant additional features as compared to conventional or known computers, processes, or the like, as discussed in greater detail herein. Embodiments within the scope of the present disclosure also include computer-readable media for carrying or having computer-executable instructions or data structures stored thereon. Such computer-readable media can be any available media that can be accessed by a computer, or downloadable through communication networks. By way of example, and not limitation, such computer-readable media can comprise various forms of data storage devices or media such as RAM, ROM, flash memory, EEPROM, CD-ROM, DVD, or other optical disk storage, magnetic disk storage, solid state drives (SSDs) or other data storage devices, any type of removable non-volatile memories such as secure digital (SD), flash memory, memory stick, etc., or any other medium which can be used to carry or store computer program code in the form of computer-executable instructions or data structures and which can be accessed by a general purpose computer, special purpose computer, specially-configured computer, mobile device, etc.

When information is transferred or provided over a network or another communications connection (either hardwired, wireless, or a combination of hardwired or wireless) to a computer, the computer properly views the connection as a computer-readable medium. Thus, any such connection is properly termed and considered a computer-readable medium. Combinations of the above should also be included within the scope of computer-readable media. Computer-executable instructions comprise, for example, instructions and data that cause a general-purpose computer, special purpose computer, or special purpose processing device such as a mobile device processor to perform one specific function or a group of functions.

Those skilled in the art will understand the features and aspects of a suitable computing environment in which aspects of the disclosure may be implemented. Although not required, some of the embodiments of the claimed systems may be described in the context of computer-executable instructions, such as program modules or engines, as described earlier, being executed by computers in networked environments. Such program modules are often reflected and illustrated by flow charts, sequence diagrams, exemplary screen displays, and other techniques used by those skilled in the art to communicate how to make and use such computer program modules. Generally, program modules include routines, programs, functions, objects, components, data structures, application programming interface (API) calls to other computers whether local or remote, etc. that perform particular tasks or implement particular defined data types, within the computer. Computer-executable instructions, associated data structures and/or schemas, and program modules represent examples of the program code for executing steps of the methods disclosed herein. The particular sequence of such executable instructions or associated data structures represent examples of corresponding acts for implementing the functions described in such steps.

Those skilled in the art will also appreciate that the claimed and/or described systems and methods may be practiced in network computing environments with many types of computer system configurations, including personal computers, smartphones, tablets, hand-held devices, multi-processor systems, microprocessor-based or programmable consumer electronics, networked PCs, minicomputers, mainframe computers, and the like. Embodiments of the claimed system are practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing various aspects of the described operations, which is not illustrated, includes a computing device including a processing unit, a system memory, and a system bus that couples various system components including the system memory to the processing unit. The computer will typically include one or more data storage devices for reading data from and writing data to. The data storage devices provide nonvolatile storage of computer-executable instructions, data structures, program modules, and other data for the computer.

Computer program code that implements the functionality described herein typically comprises one or more program modules that may be stored on a data storage device. This program code, as is known to those skilled in the art, usually includes an operating system, one or more application programs, other program modules, and program data. A user may enter commands and information into the computer through keyboard, touch screen, pointing device, a script containing computer program code written in a scripting language or other input devices (not shown), such as a microphone, etc. These and other input devices are often connected to the processing unit through known electrical, optical, or wireless connections.

The computer that effects many aspects of the described processes will typically operate in a networked environment using logical connections to one or more remote computers or data sources, which are described further below. Remote computers may be another personal computer, a server, a router, a network PC, a peer device or other common network node, and typically include many or all of the elements described above relative to the main computer system in which the systems are embodied. The logical connections between computers include a local area network (LAN), a wide area network (WAN), virtual networks (WAN or LAN), and wireless LANs (WLAN) that are presented here by way of example and not limitation. Such networking environments are commonplace in office-wide or enterprise-wide computer networks, intranets, and the Internet.

When used in a LAN or WLAN networking environment, a computer system implementing aspects of the system is connected to the local network through a network interface or adapter. When used in a WAN or WLAN networking environment, the computer may include a modem, a wireless link, or other mechanisms for establishing communications over the wide area network, such as the Internet. In a networked environment, program modules depicted relative to the computer, or portions thereof, may be stored in a remote data storage device. It will be appreciated that the network connections described or shown are exemplary and other mechanisms of establishing communications over wide area networks or the Internet may be used.

While various aspects have been described in the context of a preferred embodiment, additional aspects, features, and methodologies of the claimed systems will be readily discernible from the description herein, by those of ordinary skill in the art. Many embodiments and adaptations of the disclosure and claimed systems other than those herein described, as well as many variations, modifications, and equivalent arrangements and methodologies, will be apparent from or reasonably suggested by the disclosure and the foregoing description thereof, without departing from the substance or scope of the claims. Furthermore, any sequence(s) and/or temporal order of steps of various processes described and claimed herein are those considered to be the best mode contemplated for carrying out the claimed systems. It should also be understood that, although steps of various processes may be shown and described as being in a preferred sequence or temporal order, the steps of any such processes are not limited to being carried out in any particular sequence or order, absent a specific indication of such to achieve a particular intended result. In most cases, the steps of such processes may be carried out in a variety of different sequences and orders, while still falling within the scope of the claimed systems. In addition, some steps may be carried out simultaneously, contemporaneously, or in synchronization with other steps.

Aspects, features, and benefits of the claimed devices and methods for using the same will become apparent from the information disclosed in the exhibits and the other applications as incorporated by reference. Variations and modifications to the disclosed systems and methods may be effected without departing from the spirit and scope of the novel concepts of the disclosure.

It will, nevertheless, be understood that no limitation of the scope of the disclosure is intended by the information disclosed in the exhibits or the applications incorporated by reference; any alterations and further modifications of the described or illustrated embodiments, and any further applications of the principles of the disclosure as illustrated therein are contemplated as would normally occur to one skilled in the art to which the disclosure relates.

The foregoing description of the exemplary embodiments has been presented only for the purposes of illustration and description and is not intended to be exhaustive or to limit the devices and methods for using the same to the precise forms disclosed. Many modifications and variations are possible in light of the above teaching.

The embodiments were chosen and described in order to explain the principles of the devices and methods for using the same and their practical application so as to enable others skilled in the art to utilize the devices and methods for using the same and various embodiments and with various modifications as are suited to the particular use contemplated. Alternative embodiments will become apparent to those skilled in the art to which the present devices and methods for using the same pertain without departing from their spirit and scope. Accordingly, the scope of the present devices and methods for using the same is defined by the appended claims rather than the foregoing description and the exemplary embodiments described therein.

What is claimed is:

1. A method, comprising:
receiving, via at least one computing device, a request to generate a scheduling object from a particular network address, the request comprising a subset of a plurality of criteria;
determining, via the at least one computing device, a plurality of entries based on the subset of the plurality of criteria;
generating, via the at least one computing device, at least one respective match score for each of the plurality of entries;
generating, via the at least one computing device, a ranking of the plurality of entries based on each corresponding one of the at least one respective match score;
determining, via the at least one computing device, one or more top-ranked entries based on the ranking;
serving, via the at least one computing device, the one or more top-ranked entries to the particular network address;
receiving, via the at least one computing device, a selection of a particular entry of the one or more top-ranked entries;
in response to the selection, generating, via the at least one computing device, the scheduling object based on the particular entry;
transmitting, via the at least one computing device, the scheduling object to the particular network address; and
storing, via the at least one computing device, the scheduling object in a storage device associated with the at least one computing device.

2. The method of claim 1, wherein the plurality of criteria comprises one or more of a time interval, a location, one or more treatment preferences, and one or more product preferences.

3. The method of claim 2, wherein the one or more treatment preferences comprise at least one treatment preference selected from the group consisting of: practitioner age, practitioner gender, treatment, treatment intensity, and treatment duration.

4. The method of claim 1, wherein the subset of the plurality of criteria comprises a location of the at least one computing device.

5. The method of claim 1, further comprising, in response to detecting an access event, determining a location of the at least one computing device, wherein at least one of the plurality of criteria is based on the location.

6. The method of claim 1, wherein determining the plurality of entries comprises identifying, for each of the plurality of entries:
a particular facility from a set of facilities;
a particular practitioner from a set of practitioners corresponding to the particular facility; and
a particular treatment from a set of treatments corresponding to the particular practitioner, wherein at least one of the particular facility, the particular practitioner, or the particular treatment satisfies at least one of the subset of the plurality of criteria.

7. The method of claim 1, wherein generating the at least one match score for each of the plurality of entries comprises:
determining, for each of the plurality of entries; a plurality of historical ratings; and
combining the plurality of historical ratings to generate the at least one match score.

8. The method of claim 7, wherein combining the plurality of historical ratings comprises:
applying a weight value to each of the plurality of historical ratings to generate a plurality of weighted ratings; and
averaging the plurality of weighted ratings to generate the at least one match score.

9. The method of claim 8, further comprising training and executing at least one machine learning model to estimate the weight value of each of the plurality of historical ratings.

10. A system, comprising:
at least one data store; and
at least one computing device operatively connected to the at least one data store, wherein the at least one computing device is configured to:
receive, from a second computing device, a request to generate a scheduling object from a particular network address, the request comprising a subset of a plurality of criteria;
determine a plurality of entries based on the subset of the plurality of criteria;
generate at least one respective match score for each of the plurality of entries;
generate a ranking of the plurality of entries based on each corresponding one of the at least one respective match score;
determine one or more top-ranked entries based on the ranking;
serve the one or more top-ranked entries to the particular network address;
receive a selection of a particular entry of the one or more top-ranked entries;
in response to the selection, generate the scheduling object based on the particular entry;
transmit the scheduling object to the particular network address; and
store the scheduling object at the at least one data store.

11. The system of claim 10, wherein the plurality of criteria comprises one or more of a time interval, a location, one or more treatment preferences, and one or more product preferences.

12. The system of claim 11, wherein the one or more treatment preferences comprise at least one treatment preference selected from the group consisting of: practitioner age, practitioner gender, treatment, treatment intensity, and treatment duration.

13. The system of claim 10, wherein the subset of the plurality of criteria comprises a location of the second computing device.

14. The system of claim 10, wherein the at least one computing device is configured to, in response to detecting an access event, determine a location of the second computing device, wherein at least one of the plurality of criteria is based on the location.

15. A non-transitory computer-readable storage medium storing a plurality of instructions executable by at least one processor, wherein the plurality of instructions, when executed by the at least one processor, cause the at least one processor to:
receive, from a second computing device, a request to generate a scheduling object from a particular network address, the request comprising a subset of a plurality of criteria;
determine a plurality of entries based on the subset of the plurality of criteria;
generate at least one respective match score for each of the plurality of entries;
generate a ranking of the plurality of entries based on each corresponding one of the at least one respective match score;
determine one or more top-ranked entries based on the ranking;
serve the one or more top-ranked entries to the particular network address;
receive a selection of a particular entry of the one or more top-ranked entries;
in response to the selection, generate the scheduling object based on the particular entry;
transmit the scheduling object to the particular network address; and
store the scheduling object in at least one data store.

16. The non-transitory computer-readable storage medium of claim 15, wherein the plurality of instructions, when executed by the at least one processor, further cause the at least one processor to determine the plurality of entries by identifying, for each of the plurality of entries:
a particular facility from a set of facilities;
a particular practitioner from a set of practitioners corresponding to the particular facility; and
a particular treatment from a set of treatments corresponding to the particular practitioner, wherein at least one of the particular facility, the particular practitioner, or the particular treatment satisfies at least one of the subset of the plurality of criteria.

17. The non-transitory computer-readable storage medium of claim 15, wherein the plurality of instructions, when executed by the at least one processor, further cause the at least one processor to:
determine, for each of the plurality of entries; a plurality of historical ratings; and
combine the plurality of historical ratings to generate the at least one match score for each of the plurality of entries.

18. The non-transitory computer-readable storage medium of claim 17, wherein the at least one computing device is configured to combine the plurality of historical ratings by averaging the plurality of historical ratings.

19. The non-transitory computer-readable storage medium of claim 17, wherein the plurality of instructions, when executed by the at least one processor, further cause the at least one processor to combine the plurality of historical ratings by:
applying a weight value to each of the plurality of historical ratings to generate a plurality of weighted ratings; and
averaging the plurality of weighted ratings to generate the at least one match score.

20. The non-transitory computer-readable storage medium of claim 19, wherein the plurality of instructions, when executed by the at least one processor, further cause the at least one processor to train and execute at least one machine learning model to estimate the weight value of each of the plurality of historical ratings.
